SEQUENCE LISTING (14 sequences)

SEQ ID NO: 1 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 2 - protein, 742 aa, Artificial sequence
  NKAB-ErbB2
MDWIWRILFLVGAATGAHSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLASPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 3 - DNA, 2229 bp, Artificial sequence
  NKAB-ErbB2
atggactggatttggcgcatcctgttcctcgtgggagccgccaccggtgcccattctcaggtgcagctggtggaatctggcggcggactcgtgaagcctggcggctctctgagactgagctgtgccgccagcggcttcaccttcagcagctacggaatgcactgggtgcgccaggcccctggcaaaggactggaatgggtggccttcatcagatacgacggcagcaacaagtactacgccgacagcgtgaagggccggttcaccatctcccgggacaacagcaagaacaccctgtacctgcagatgaacagcctgcgggccgaggacaccgccgtgtactactgcgccaaggatagaggcctgggcgacggcacctacttcgactattggggccagggcaccaccgtgaccgtgtctagtggcggaggcggatcaggcggcggaggatcagggggagggggatctcagtctgccctgacacagcctgccagcgtgtccggatctcctggccagagcatcaccatcagctgcagcggcagcagcagcaacatcggcaacaacgccgtgaactggtatcagcagctgcccggcaaggcccccaaactgctgatctactacgacgacctgctgcccagcggcgtgtccgatagattcagcggctccaagagcggcaccagcgcctttctggccatcagcggcctgcagtctgaggacgaggccgactactattgcgccgcctgggacgacagcctgaacggccctgtgtttggaggcggcaccaagctgacagtgctggctagccccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagcctcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggcggaggcggatcaggcggcggaggatcccaggtgcagctgcagcagtctggccccgagctgaagaaacccggcgagacagtgaagatctcctgcaaggcctccggctaccccttcaccaactacggcatgaattgggtcaagcaggccccaggccagggcctgaaatggatgggctggatcaacaccagcaccggcgagagcaccttcgccgacgacttcaagggcagattcgacttcagcctggaaaccagcgccaacaccgcctatctgcagatcaacaatctgaagtccgaggacagcgctacctacttctgcgccagatgggaggtgtaccacggctacgtgccatactggggacagggaacaacagtgacagtgtcctctggcgggggaggaagtggggggggaggatctgggggcggaggcagtgatatccagctgacccagagccacaagtttctgagcaccagcgtgggcgaccgggtgtccatcacctgtaaagccagccaggacgtgtacaatgccgtggcttggtatcagcagaagcctggccagagccctaaactgctgatctatagcgccagcagccggtacaccggcgtgccctctagattcaccggatctggcagcggccctgacttcacctttaccatctccagcgtgcaggccgaagatctggccgtgtatttctgccagcagcacttccggacccctttcacctttggctccggcacaaagctggaaatcaaatga

SEQ ID NO: 4 - protein, 966 aa, Artificial sequence
  NKAB-ErbB2_RD-IL15
MDWIWRILFLVGAATGAHSITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSGGGSSGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLASPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSQVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 5 - protein, 241 aa, Artificial sequence
  anti-CD19 scFv
EVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEL

SEQ ID NO: 6 - protein, 245 aa, Artificial sequence
  anti-CD20 scFv
QVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKRA

SEQ ID NO: 7 - protein, 241 aa, Artificial sequence
  anti-EGFR scFv
QVQLQESGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTTVTVSSGGGGSGGGGSGGGGSDIQLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLEIK

SEQ ID NO: 8 - protein, 240 aa, Artificial sequence
  anti-PD-L1 scFv
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 9 - protein, 371 aa, Artificial sequence
  NKG2D-CAR (NKAR)
MDWIWRILFLVGAATGAHSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSEQKLISEEDLALSNSIMYFSHFVPVFLPAKPTTTPAPRPPTPAPTIASQPLSLRPEASRPAAGGAVHTRGLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1116 bp, Artificial sequence
  NKG2D-CAR (NKAR)
atggattggatctggcggatcctgttcctcgtgggagccgccacaggcgcccacagcctgttcaatcaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtgggcggaggcggaagtggcggcggaggatctgagcagaagctgatctccgaagaggacctggccctgagcaacagcatcatgtacttcagccacttcgtgcccgtgtttctgcccgccaagcctaccacaaccccagcccctagacctcctacacccgcccctacaatcgccagccagcctctgtctctgaggcccgaggcttctagacctgctgcaggcggagctgtgcacaccaggggcctggaccccaagctgtgctacctgctggacggcatcctgttcatctacggcgtgatcctgaccgccctgttcctgagagtgaagttcagccgcagcgccgacgcccctgcctaccagcagggccagaaccagctgtacaacgagctgaacctgggcaggcgggaggaatacgacgtgctggacaagcgcagaggccgggaccctgagatgggcggcaagcccaggcggaagaacccccaggaaggcctgtataacgaactgcagaaagacaagatggccgaggcctacagcgagatcggcatgaagggcgagcggcgacgcggcaagggccacgacggcctgtaccagggcctgtccaccgccaccaaggacacctacgacgccctgcacatgcaggccctgcctccccgttaa

SEQ ID NO: 11 - protein, 418 aa, Artificial sequence
  KG2D-CAR with CD28 costimulatory sequence
MDWIWRILFLVGAATGAHSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSEQKLISEEDLALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1257 bp, Artificial sequence
  NKG2D-CAR with CD28 costimulatory sequence
atggattggatctggcggatcctgttcctcgtgggagccgccacaggcgcccacagcctgttcaatcaggaagtgcagatccccctgaccgagagctactgcggcccctgccccaagaactggatctgctacaagaacaactgctaccagttcttcgacgagagcaagaattggtacgagagccaggccagctgcatgagccagaacgccagcctgctgaaggtgtacagcaaagaggaccaggatctgctgaagctcgtgaagtcctaccactggatgggcctggtgcacatccccaccaatggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgtatgcagcggaccgtgggcggaggcggaagtggcggcggaggatctgagcagaagctgatctccgaagaggacctggccctgagcaacagcatcatgtacttcagccacttcgtgcccgtgtttctgcccgccaagcctaccacaaccccagcccctagacctcctacacccgcccctacaatcgccagccagcctctgtctctgaggcccgaggcttctagacctgctgcaggcggagctgtgcacaccaggggcctggacaagcccttctgggtgctggtcgtggtcggcggagtgctggcctgttacagcctgctggtcaccgtggccttcatcatcttttgggtccgcagcaagcggagccggctgctgcacagcgactacatgaacatgaccccaaggcggccaggccccacccggaagcactaccagccctatgcccctcctagggacttcgccgcctaccggtccagagtgaagttcagccgcagcgccgacgcccctgcctaccagcagggccagaaccagctgtacaacgagctgaacctgggcaggcgggaggaatacgacgtgctggacaagcgcagaggccgggaccctgagatgggcggcaagcccaggcggaagaacccccaggaaggcctgtataacgaactgcagaaagacaagatggccgaggcctacagcgagatcggcatgaagggcgagcggcgacgcggcaagggccacgacggcctgtaccagggcctgtccaccgccaccaaggacacctacgacgccctgcacatgcaggccctgcctccccgttaa

SEQ ID NO: 13 - protein, 230 aa, Artificial sequence
  IL-15 agonist
MDWIWRILFLVGAATGAHSITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 14 - DNA, 693 bp, Artificial sequence
  IL-15 agonist (RD-IL-15)
atggactggatttggcgcatcctgttcctcgtgggagccgccaccggtgcccattctatcacctgtcctccacctatgagcgtggaacacgccgacatctgggtcaagagctacagcctgtacagcagagagcggtacatctgcaacagcggcttcaagagaaaggccggcaccagcagcctgaccgagtgtgtgctgaacaaggccaccaatgtagcccactggaccacacctagcctgaagtgcatcagagatcccgctctggtgcatcagcgacctgctccacctggcggaggatctggtggtggtggaagcggaggcggatctggcggcggaggttctctgcagaattgggtcaacgtgatctccgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacgacagcctgagcagcaacggcaatgtgaccgagtccggctgcaaagagtgcgaggaactggaagagaagaatatcaaagagttcctgcagagcttcgtgcacatcgtgcagatgttcatcaacaccagctga

(19) United States
(12) Patent Application Publication
(10) Pub. No.: US 2024/0174768 A1
(43) Pub. Date: May 30, 2024

(54) BISPECIFIC ANTIBODIES ENHANCING CELL MEDIATED IMMUNE RESPONSES

(71) Applicant: CHEMOTHERAPEUTISCHES FORSCHUNGSINSTITUT GEORG-SPEYER-HAUS, Frankfurt am Main (DE)

(72) Inventors: Winfried WELS, Frankfurt am Main (DE); Congcong ZHANG, Bergisch Gladbach (DE); Pranav OBEROI, Oslo (NO); Anne KIEFER, Frankfurt (DE)

(21) Appl. No.: 18/280,104

(22) PCT Filed: Mar. 3, 2022
(86) PCT No.: PCT/EP2022/055438

§ 371 Date: Sep. 1, 2023

(30) Foreign Application Priority Data
Mar. 3, 2021 (EP) 21160548.0

(51) Int. Cl. C07K 16/46; A61K 39/00; A61P 35/00; C07K 14/54

(52) U.S. Cl. CPC C07K 16/468; A61K 39/4631; A61P 35/00; C07K 14/5443; A61K 2239/13; A61K 2239/17; A61K 2239/21; A61K 2239/22; C07K 2317/31

(57) ABSTRACT

The invention pertains to bispecific antibodies having two antigen binding specificities, one binding to an epitope of NKG2-D type II integral membrane protein (NKG2D) and one binding to an antigen associated with a disease, preferably a tumor associated- or tumor specific antigen, such as ErbB2 (HER2), CD19, CD20, GD2, PD-L1, EGFR, or EGFRvIII. The bispecific molecules of the invention are preferably applied in the context of the treatment of tumor diseases or infectious diseases. Surprisingly it was found that the use of NKG2D binding specificities that bind in a competitive manner to the NKG2D receptor with its natural ligands such as MICA reduces or prevents the inhibitory effect of ligand shedding. Another advantage of the present invention lies in a synergistic combination of the bispecific molecules of the invention and chimeric antigen receptor (CAR) based therapy. Further provided are methods for the production of the antibodies of the invention, nucleic acids encoding the bispecific antibodies or fragments thereof, pharmaceutical composition and recombinant cells comprising nucleic acids or antibody proteins.

### FIELD OF THE INVENTION

The invention pertains to bispecific antibodies having two antigen binding specificities, one binding to an epitope of NKG2-D type II integral membrane protein (NKG2D) and one binding to an antigen associated with a disease, preferably a tumor associated- or tumor specific antigen, such as ErbB2 (HER2), CD19, CD20, GD2, PD-L1, EGFR, or EGFRvIII. The bispecific molecules of the invention are preferably applied in the context of the treatment of tumor diseases or infectious diseases. Surprisingly it was found that the use of NKG2D binding specificities that bind in a competitive manner to the NKG2D receptor with its natural ligands such as MICA reduces or prevents the inhibitory effect of ligand shedding. Another advantage of the present invention lies in a synergistic combination of the bispecific molecules of the invention and chimeric antigen receptor (CAR) based therapy. Further provided are methods for the production of the antibodies of the invention, nucleic acids encoding the bispecific antibodies or fragments thereof, pharmaceutical composition and recombinant cells comprising nucleic acids or antibody proteins.

### DESCRIPTION

Precision cancer immunotherapy with chimeric antigen receptor (CAR)-engineered T cells has demonstrated remarkable clinical efficacy in patients with B-cell malignancies (June, C. H., O'Connor, R. S., Kawalekar, O. U., Ghassemi, S. & Milone, M. C. CAR T cell immunotherapy for human cancer. Science 359, 1361-1365 (2018)). However, successful targeting of more prevalent solid tumors with CAR-T cells still remains a challenge, owing in part to an immunosuppressive tumor microenvironment, limited persistence of infused effector cells, and intra-tumoral heterogeneity of target antigen expression (Gad, A. Z., El-Naggar, S. & Ahmed, N. Realism and pragmatism in developing an effective chimeric antigen receptor T-cell product for solid cancers. Cytotherapy 18, 1382-1392 (2016) and DeRenzo, C., Krenciute, G. & Gottschalk, S. The landscape of CAR T cells beyond acute lymphoblastic leukemia for pediatric solid tumors. Am Soc Clin Oncol Educ Book 38, 830-837 (2018)). With respect to the latter, current CAR effector cells typically recognize a single tumor-associated surface antigen, resulting in only limited cell killing in tumors with uneven distribution of the target molecule, and favoring outgrowth of sub-clones with low or absent antigen expression (Sotillo, E. et al. Convergence of acquired mutations and alternative splicing of CD19 enables resistance to CART-19 immunotherapy. Cancer Discov 5, 1282-1295 (2015) and Genßler, S. et al. Dual targeting of glioblastoma with chimeric antigen receptor-engineered natural killer cells overcomes heterogeneity of target antigen expression and enhances antitumor activity and survival. Oncoimmunology 5, e1119354 (2016) and O'Rourke, D. M. et al. A single dose of peripherally infused EGFRvIII-directed CAR T cells mediates antigen loss and induces adaptive resistance in patients with recurrent glioblastoma. Sci Transl Med 9 (2017)). Hence, different strategies are being followed to enable recognition of multiple target antigens, which include combination of two distinct monospecific CAR-T cells and co-expression of two CARs in the same T cells (Ruella, M. et al. Dual CD19 and CD123 targeting prevents antigen-loss relapses after CD19-directed immunotherapies. J Clin Invest 126, 3814-3826 (2016)), or the more complex generation of bivalent and trivalent CARs harboring more than one cell-binding domain within a single molecule (Grada, Z. et al. TanCAR: A novel bispecific chimeric antigen receptor for cancer immunotherapy. Mol Ther Nucleic Acids 2, e105 (2013) and Bielamowicz, K. et al. Trivalent CAR T cells overcome interpatient antigenic variability in glioblastoma. Neuro Oncol 20, 506-518 (2018)).

Another approach harnesses the broad tumor specificity of the Natural Killer Group 2D (NKG2D) molecule in a CAR design, linking the extracellular ligand-binding domain of NKG2D to a heterologous signaling molecule such as CD3ζ (Zhang, T., Lemoi, B. A. & Sentman, C. L. Chimeric NK-receptor-bearing T cells mediate antitumor immunotherapy. Blood 106, 1544-1551 (2005) and Spear, P., Wu, M. R., Sentman, M. L. & Sentman, C. L. NKG2D ligands as therapeutic targets. Cancer Immun 13, 8 (2013) and Lazarova, M., Wels, W. S. & Steinle, A. Arming cytotoxic lymphocytes for cancer immunotherapy by means of the NKG2D/NKG2D-ligand system. Expert Opin Biol Ther (2020) and Obajdin, J., Davies, D. M. & Maher, J. Engineering of chimeric natural killer cell receptors to develop precision adoptive immunotherapies for cancer. Clin Exp Immunol (2020)). Natural NKG2D is an activating receptor expressed by all NK cells, CD8+ T cells, and most natural killer T (NKT) cells, as well as subpopulations of CD4+ T cells and γδ T cells (Lazarova, M., Wels, W. S. & Steinle, A. Arming cytotoxic lymphocytes for cancer immunotherapy by means of the NKG2D/NKG2D-ligand system. Expert Opin Biol Ther (2020) and Raulet, D. H., Gasser, S., Gowen, B. G., Deng, W. W. & Jung, H. Y. Regulation of ligands for the NKG2D activating receptor. Annu Rev Immunol 31, 413-441 (2013) and Lanier, L. L. NKG2D receptor and its ligands in host defense. Cancer Immunol Res 3, 575-582 (2015)). NKG2D has multiple membrane-anchored ligands widely expressed in virus-infected cells (Zingoni et al. NKG2D and its ligands: “One for all, all for one”. Front Immunol 9, 476 (2018)) and almost all cancer types, such as lung, breast, kidney, pancreatic, ovarian and prostate cancer, melanoma, leukemia and glioblastoma (Ullrich, E., Koch, J., Cerwenka, A. & Steinle, A. New prospects on the NKG2D/NKG2DL system for oncology. Oncoimmunology 2, e26097 (2013) and Le Bert, N. & Gasser, S. Advances in NKG2D ligand recognition and responses by NK cells. Immunol Cell Biol 92, 230-236 (2014)). General safety of autologous NKG2D-CAR T cells has recently been demonstrated in a phase I trial in patients with acute myeloid leukemia/myelodysplastic syndrome or multiple myeloma (Nikiforow, S. et al. Safety data from a first-in-human phase 1 trial of NKG2D chimeric antigen receptor-T cells in AML/MDS and multiple myeloma. Blood 128 (2016) and Baumeister, S. H. et al. Phase I trial of autologous CAR T cells targeting NKG2D ligands in patients with AML/MDS and multiple myeloma. Cancer Immunol Res 7, 100-112 (2019)). Nevertheless, such NKG2D-CAR cells will likely not be active against leukemic stem cells with low or absent expression of NKG2D ligands (Paczulla, A. M. et al. Absence of NKG2D ligands defines leukaemia stem cells and mediates their immune evasion. Nature (2019)). Natural NKG2D-mediated immune surveillance can be counteracted through a variety of mechanisms, including downregulation of NKG2D ligands upon exposure of cancer cells to IFN-γ or TGF-β (Schwinn, N. et al. Interferon-gamma down-regulates NKG2D ligand expression and impairs the NKG2D-mediated cytolysis of MHC class I-deficient melanoma by natural killer cells. Int J Cancer 124, 1594-1604 (2009) and Eisele, G. et al. TGF-beta and metalloproteinases differentially suppress NKG2D ligand surface expression on malignant glioma cells. Brain 129, 2416-2425 (2006)). Tumor cells can also decrease NKG2D ligand density on the cell surface by proteolytic shedding (Salih, H. R., Rammensee, H. G. & Steinle, A. Cutting edge: down-regulation of MICA on human tumors by proteolytic shedding. J Immunol 169, 4098-4102 (2002)). Accordingly, in the sera of cancer patients elevated levels of soluble NKG2D ligands have been found, in many cases correlating with disease stage and metastasis (Le Bert, N. & Gasser, S. Advances in NKG2D ligand recognition and responses by NK cells. Immunol Cell Biol 92, 230-236 (2014) and Raulet, D. H., Gasser, S., Gowen, B. G., Deng, W. W. & Jung, H. Y. Regulation of ligands for the NKG2D activating receptor. Annu Rev Immunol 31, 413-441 (2013)). High concentrations of shedded NKG2D ligands can act as a competitor and block the interaction of NKG2D with tumor cells or virus infected cells, induce NKG2D internalization and degradation, and desensitize immune effector cells (Raulet, D. H., Gasser, S., Gowen, B. G., Deng, W. W. & Jung, H. Y. Regulation of ligands for the NKG2D activating receptor. Annu Rev Immunol 31, 413-441 (2013), and Salih, H. R., Rammensee, H. G. & Steinle, A. Cutting edge: down-regulation of MICA on human tumors by proteolytic shedding. J Immunol 169, 4098-4102 (2002) and Groh, V., Wu, J., Yee, C. & Spies, T. Tumour-derived soluble MIC ligands impair expression of NKG2D and T-cell activation. Nature 419, 734-738 (2002) and Groth, A., Kloss, S., von Strandmann, E. P., Koehl, U. & Koch, J. Mechanisms of tumor and viral immune escape from natural killer cell-mediated surveillance. J Innate Immun 3, 344-354 (2011).

Raynaud et al. disclose anti-human NKG2D single-domain antibodies integrated into bivalent and bispecific formats showing activating or inhibitory effects on the immune response mediated by the NKG2DL/NKG2D axis (Raynaud, A. et al. Anti-NKG2D single domain-based antibodies for the modulation of anti-tumor immune response. Onoimmunology 10, 1854529 (2021)). The bispecific antibodies of Raynaud et al. that do not compete with ligand binding retained their full cytotoxic activity and are suggested as therapeutics to circumvent immunosuppressive effects. However, bispecific antibodies according to the teachings by Raynaud et al. that target a tumor antigen and NKG2D as an activating receptor were in all cases less effective than a similar bispecific antibody that targets the same tumor antigen and CD16 as an activating receptor.

WO 2019/178576 discloses the treatment of cancer using a bispecific antibody construct that presents one binding specificity for NKG2D, and a second binding specificity for an antigen target expressed on tumor cells in combination with a chimeric antigen receptor comprising an antigen binding domain of an antibody specific for a second tumor-associated antigen. The document does not teach specific bispecific formats, nor a combined use of a bispecific NKG2D antibody with a chimeric antigen receptor that comprises an extracellular NKG2D sequence through which it can directly interact with said bispecific NKG2D antibody.

US 2021/032349 A1 discloses bispecific antibodies characterised by one binding site that specifically binds to NKG2D and the other binding site that specifically binds to CEA (carcinoembryonic antigen).

US 2019/375838 A1 discloses bispecific antibodies characterized by one binding site that specifically binds to NKG2D and the other binding site that specifically binds to BCMA (B cell maturation antigen).

Thus, it is an objection of the invention to provide an antibody based therapeutic molecule which overcomes the aforementioned drawbacks known in the art.

### BRIEF DESCRIPTION OF THE INVENTION

To limit potential interference by soluble NKG2D ligands and redirect NKG2D expressing immune cells specifically to defined target cells, the inventors designed bispecific antibodies combining in a single tetravalent molecule two NKG2D-binding and two single chain fragment variable (scFv) domains specific for a target antigen of interest, such as ErbB2 (HER2), CD19, CD20, GD2, PD-L1, EGFR, or EGFRvIII, with the NKG2D-binding and target antigen-binding domains linked by the hinge, CH2 and CH3 regions of IgG4 or IgG1. For such an NKAB-ErbB2 antibody, specific targeting to ErbB2-positive breast carcinoma cells and enhancement of NKG2D-mediated cytotoxicity was evaluated in in vitro cell killing experiments with peripheral blood derived unsorted primary lymphocytes and purified primary natural killer (NK) cells endogenously expressing NKG2D, and established NK-92 cells as a further example for NK cells and primary T cells which carry an NKG2D-based chimeric activating receptor (NKAR) encompassing the extracellular domain of NKG2D fused to transmembrane and intracellular domains of CD3ζ. Combined antitumor effects of the NKAB-ErbB2 molecule and the NKAR-NK cells in vivo were investigated in a syngeneic tumor model in immunocompetent C57BL/6 mice with ErbB2-positive glioblastoma cells. To test general applicability of this approach, for NKAB-CD19 and NKAB-CD20 antibodies specific targeting to CD19- and CD20-positive lymphoma cells and combined antitumor effects of NKAB-CD19 and NKAB-CD20 antibodies and the NKAR-NK cells were investigated in in vitro cell killing experiments.

Therefore, and by way of brief description, the main aspects based on the above results of the present invention can be described as follows:

In a first aspect, the invention pertains to a binding molecule which is at least bispecific comprising at least a first and a second binding domain for use in the treatment of a disease in a subject, wherein
the first binding domain is capable of binding to an epitope of NKG2-D type II integral membrane protein (NKG2D, SEQ ID NO: 1); and
the second binding domain is capable of binding to an epitope of an antigenic target protein expressed on or in a cell associated with the disease in the subject;
   wherein the treatment comprises an administration of the binding molecule to the subject and an administration of an immune cell expressing NKG2D to the subject.

In a second aspect, the invention pertains to a binding molecule which is at least bispecific comprising at least a first and a second binding domain, wherein
the first binding domain is capable of binding to an epitope of NKG2-D type II integral membrane protein (NKG2D, SEQ ID NO: 1); and
the second binding domain is capable of binding to an epitope of an antigenic target protein expressed on or in a cell associated with the disease in the subject;
   wherein the first and the second binding domain are antibody scFv constructs and are linked to each other via human IgG1 or IgG4 derived hinge, CH2 and CH3 domains.

In a third aspect, the invention pertains to an immune cell expressing an immune cell receptor, or an immune cell receptor, for use in the treatment of a disease in a subject, wherein the immune cell receptor is NKG2D, or a derivative thereof such as a chimeric antigen receptor (CAR), and wherein the immune cell optionally further expresses an interleukin-15, or an interleukin-15 agonist, the treatment comprising an administration of a binding molecule recited in the first or second aspect, and an administration of the immune cell or the immune cell receptor to the subject.

In a fourth aspect, the invention pertains to an isolated nucleic acid, comprising one or more sequences encoding for a binding molecule recited in the first or second aspect of the invention.

In a fifth aspect, the invention pertains to a recombinant host cell, comprising a nucleic acid of the fourth aspect.

In a sixth aspect, the invention pertains to a pharmaceutical composition or package comprising:
(i) A binding molecule of the first or second aspect; or (ii) a nucleic acid, or a recombinant host cell of an aspect of the invention; and/or
an immune cell expressing the immune cell receptor, or the immune cell receptor, recited in the third aspect;
   together with a pharmaceutically acceptable carrier, stabilizer and/or excipient.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a binding molecule which is at least bispecific comprising at least a first and a second binding domain for use in the treatment of a disease in a subject, wherein
the first binding domain is capable of binding to an epitope of NKG2-D type II integral membrane protein (NKG2D, SEQ ID NO: 1); and
the second binding domain is capable of binding to an epitope of an antigenic target protein expressed on or in a cell associated with the disease in the subject;
   wherein the treatment comprises an administration of the binding molecule to the subject and an administration of an immune cell expressing NKG2D to the subject.

In a second aspect, the invention pertains to a binding molecule which is at least bispecific comprising at least a first and a second binding domain, wherein
the first binding domain is capable of binding to an epitope of NKG2-D type II integral membrane protein (NKG2D, SEQ ID NO: 1); and
the second binding domain is capable of binding to an epitope of an antigenic target protein expressed on or in a cell associated with the disease in the subject;
   wherein the first and the second binding domain are antibody scFv constructs and are linked to each other via human IgG1 or IgG4 derived hinge, CH2 and CH3 domains.

The term “NKG2-D type II integral membrane protein” or “NKG2D”, pertains to a protein encoded by the gene KLRK1. The protein functions as an activating and costimulatory receptor involved in immunosurveillance upon binding to various cellular stress-inducible ligands displayed at the surface of autologous tumor cells and virus-infected cells. NKG2D provides both stimulatory and costimulatory innate immune responses on activated natural killer (NK) and NKT cells, leading to cytotoxic activity, and it may act as a stimulatory and costimulatory receptor in CD8+ and CD4+ T-cell-mediated adaptive immune responses by amplifying T-cell activation. It's signaling cascade involves calcium influx, culminating in the expression of TNF-α. Known ligands of NKG2D belong to various subfamilies of MHC class I-related glycoproteins including MICA, MICB, ULBP1/RAET1, ULBP2/RAET1H, ULBP3/RAET1N, ULBP4/RAET1E, ULBP5/RAET1G and ULBP6/RAET1L. More information on the protein can be derived from the UniProt database (“www.uniprot.org”) in the database version of Feb. 1, 2021, under the accession number P26718. The human protein amino acid sequence is provided herein below as SEQ ID NO: 1.

The term “Interleukin-15” or “IL-15” refers to a protein with stimulatory effects towards the proliferation of T lymphocytes. The protein or encoding nucleic acid sequence can be derived from the UniProt database (“www.uniprot.org”) in the database version of Feb. 1, 2021, under the accession number P40933 (human Interleukin-15).

A binding molecule of the invention is preferably an antigen binding protein (ABP).

An “antigen binding protein” (“ABP”) as used herein means that one or more binding sites of the binding molecule of the invention are provided by a protein that specifically binds to a target antigen, such as to one or more epitope(s) displayed by or present on a target antigen. One central antigen of the ABPs of the invention is NKG2D or an orthologue (or paralogue) or other variant thereof; and the ABP can, optionally bind to one or more domains of said NKG2D or variant (such as the epitope(s) can be displayed by or presented on one or more extracellular domains of said NKG2D or variant); preferably the ABP of the invention binds to an epitope of NKG2D that shields the receptor from soluble or shedded forms of its natural ligands. Typically, an antigen binding protein is an antibody (or a fragment thereof), however other forms of antigen binding protein are also envisioned by the invention. For example, the ABP may be a natural ligand of NKG2D, or another (non-antibody) receptor protein derived from small and robust non-immunoglobulin “scaffolds”, such as those equipped with binding functions for example by using methods of combinatorial protein design (Gebauer & Skerra, 2009; Curr Opin Chem Biol, 13:245). Particular examples of such non-antibody ABPs include: Affibody molecules based on the Z domain of Protein A (Nygren, 2008; FEBS J 275:2668); Affilins based on gamma-B crystalline and/or ubiquitin (Ebersbach et al, 2007; J Mo Biol, 372:172); Affimers based on cystatin (Johnson et al, 2012; Anal Chem 84:6553); Affitins based on Sac7d from Sulfolobus acidcaldarius (Krehenbrink et al, 2008; J Mol Biol 383:1058); Alphabodies based on a triple helix coiled coil (Desmet et al, 2014; Nature Comms 5:5237); Anticalins based on lipocalins (Skerra, 2008; FEBS J 275:2677); Avimers based on A domains of various membrane receptors (Silverman et al, 2005; Nat Biotechnol 23:1556); DARPins based on an ankyrin repeat motif (Strumpp et al, 2008; Drug Discov Today, 13:695); Fynomers based on an SH3 domain of Fyn (Grabulovski et al, 2007; J Biol Chem 282:3196); Kunitz domain peptides based on Kunitz domains of various protease inhibitors (Nixon et al, Curr opin Drug Discov Devel, 9:261) and Centyrins and Monobodies based on a 10th type III domain of fibronectin (Diem et al., 2014; Protein Eng Des Sel 27:419 doi:10.1093/protein/gzu016; Koide & Koide, 2007; Methods Mol Biol 352:95).

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that bind the antigen binding protein (such as via an antigen binding domain of said protein). Epitope determinants can include chemically active surface groupings of molecules such as amino acids, sugar side chains, phosphoryl or sulfonyl groups, and can have specific three-dimensional structural characteristics, and/or specific charge characteristics. Generally, antigen binding proteins specific for a particular target antigen will preferentially recognize an epitope on the target antigen in a complex mixture of proteins and/or macromolecules.

An antigen binding protein is “specific” when it binds to one antigen (such as NKG2D, or a tumor associated antigen such as ErbB2) more preferentially (eg, more strongly or more extensively) than it binds to a second antigen. The term “specifically binds” (or “binds specifically” and the like) used herein in the context of an ABP means that said ABP will preferentially bind to the desired antigen than to bind to other proteins (or other molecules), such as preferentially binding to such compared to one or more of other immunoglobulin (Ig) superfamily genes. Therefore, preferably, the binding affinity of the ABP to the one antigen (e.g. NKG2D) is at least 2-fold, at least 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 10⁵-fold or even at least 10⁶-fold, most preferably at least 2-fold, compared to its affinity to the other targets (e.g. unrelated proteins such as mouse or human Fc domain, or streptavidin).

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, WI). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm.

A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may also be used by the algorithm.

Examples of parameters that can be employed in determining percent identity for polypeptides or nucleotide sequences using the GAP program are the following: (i) Algorithm: Needleman et al., 1970, J. Mol. Biol. 48:443-453; (ii) Comparison matrix: BLOSUM 62 from Henikoff et al., 1992, supra; (iii) Gap Penalty: 12 (but with no penalty for end gaps); (iv) Gap Length Penalty: 4; (v) Threshold of Similarity: 0.

A preferred method of determining similarity between a protein or nucleic acid and human NKG2D, or a binding molecule of the invention, is that provided by the Blast searches supported at Uniprot supra (e.g., http://www.uniprot.org/uniprot); in particular for amino acid identity, those using the following parameters: Program: blastp; Matrix: blosum62; Threshold: 10; Filtered: false; Gapped: true; Maximum number of hits reported: 250.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least about 10, 15, 20, 25, 30, 35, 40, 45, 50 or other number of contiguous amino acids of the target polypeptide or region thereof.

In particular embodiments, an ABP of the invention can preferentially comprise at least one complementarity determining region (CDR), such as one from an antibody (in particular from a human antibody), and in particular embodiments the ABP can comprise a CDR having an amino acid sequence with at least 80%, 85%, 90% or 95% sequence identity to (preferably, at least 90% sequence identity to), or having no more than three or two, preferably no more than one amino acid substitution(s), deletion(s) or insertion(s) compared to, a CDR sequence as comprised in an antibody sequence shown in SEQ ID NO: 2, 4, and 5 to 8.

The term “complementarity determining region” (or “CDR” or “hypervariable region”), as used herein, refers broadly to one or more of the hyper-variable or complementarily determining regions (CDRs) found in the variable regions of light or heavy chains of an antibody. See, for example: “IMGT”, Lefranc et al, 20003, Dev Comp Immunol 27:55; Honegger & Plückthun, 2001, J Mol Biol 309:657, Abhinandan & Martin, 2008, Mol Immunol 45:3832, Kabat, et al. (1987): Sequences of Proteins of Immunological Interest National Institutes of Health, Bethesda, Md. These expressions include the hypervariable regions as defined by Kabat et al (1983) Sequences of Proteins of Immunological Interest, US Dept of Health and Human Services, or the hypervariable loops in 3-dimensional structures of antibodies (Chothia and Lesk, 1987; J Mol Biol 196:901). The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. (Kashmiri, 2005; Methods 36:25).

An ABP of the invention may, alternatively or as well as a CDR3 sequence, comprise at least one CDR1, and/or at least one CDR2 (such as one from an antibody, in particular from a human antibody). Preferably, and ABP of the invention comprises at least one such CDR3, as well as at least one such CDR1 and at least one such CDR2, more preferably where each of such CDRs having an amino acid sequence with at least 80%, 85%, 90% or 95% (preferably at least 90%) sequence identity to, or having no more than three or two, preferably no more than one amino acid substitution(s), deletion(s) or insertion(s) compared to, a sequence selected from the corresponding (heavy and light chain) CDR1, CDR2 and CDR3 sequences comprised in any of the sequences shown in SEQ ID NO: 2, 4, and 5 to 8.

In particular embodiments, an ABP of the invention can be an antibody or an antigen binding fragment thereof.

As used herein, the term “antibody” may be understood in the broadest sense as any immunoglobulin (Ig) that enables binding to its epitope. An antibody as such is a species of an ABP. Full length “antibodies” or “immunoglobulins” are generally heterotetrameric glycoproteins of about 150 kDa, composed of two identical light and two identical heavy chains. Each light chain is linked to a heavy chain by one covalent disulphide bond, while the number of disulphide linkages varies between the heavy chain of different immunoglobulin isotypes. Each heavy and light chain also has regularly spaced intrachain disulphide bridges. Each heavy chain has an amino terminal variable domain (VH) followed by three carboxy terminal constant domains (CH). Each light chain has a variable N-terminal domain (VL) and a single C-terminal constant domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to cells or factors, including various cells of the immune system (e.g., effector cells) and the first component (C1q) of the classical complement system. Other forms of antibodies include heavy-chain antibodies, being those which consist only of two heavy chains and lack the two light chains usually found in antibodies. Heavy-chain antibodies include the hcIgG (IgG-like) antibodies of camelids such as dromedaries, camels, llamas and alpacas, and the IgNAR antibodies of cartilaginous fishes (for example sharks). And yet other forms of antibodies include single-domain antibodies (sdAb, called Nanobody by Ablynx, the developer) being an antibody fragment consisting of a single monomeric variable antibody domain. Single-domain antibodies are typically produced from heavy-chain antibodies, but may also be derived from conventional antibodies.

Antibodies (or those from which fragments thereof can be isolated) can include, for instance, chimeric, humanized, (fully) human, or hybrid antibodies with dual or multiple antigen or epitope specificities, antibody fragments and antibody sub-fragments, e.g., Fab, Fab′ or F(ab′)2 fragments, single chain antibodies (scFv) and the like (described below), including hybrid fragments of any immunoglobulin or any natural, synthetic or genetically engineered protein that acts like an antibody by binding to a specific antigen to form a complex.

Accordingly, in certain embodiments an ABP of the invention can comprise an antibody heavy chain, or an antigen binding fragment thereof, and/or an antibody light chain, or an antigen binding fragment thereof.

In further embodiments, an ABP of the invention can comprise an antibody heavy chain variable region, or an antigen binding fragment thereof, and/or an antibody light chain variable region, or an antigen binding fragment thereof, and in yet further embodiments, an ABP of the invention can comprise an antibody heavy chain variable region CDR1, CDR2, and CDR3, and/or an antibody light chain variable region CDR1, CDR2, and CDR3.

The present invention pertains to a binding molecule which is “bispecific” or “bifunctional”, and preferably is an ABP that has two different epitope/antigen binding domains (or “sites”), and accordingly has binding specificities for two different target epitopes. These two epitopes may be epitopes of the same antigen or, as preferred in the present invention, of different antigens, such as the different antigens as one binding to NKG2D and the other binding to a tumor associated antigen, such as ErbB2.

A “bispecific ABP”, may be an ABP that binds one antigen or epitope with one of two or more binding arms, defined by a first pair of heavy and light chain or of main and shorter/smaller chain, and binds a different antigen or epitope on a second arm, defined by a second pair of heavy and light chain or of main and smaller chain. Such an embodiment of a bispecific ABP has two distinct antigen binding arms, in both specificity and CDR sequences. Typically, a bispecific ABP is monovalent for each antigen it binds to, that is, it binds with only one arm to the respective antigen or epitope. However, bispecific antibodies can also be dimerized or multimerized, which is preferred in context of the present invention. Hence, the bispecific binding molecules of the invention are in total tetravalent. For example, in the dimeric scFv₂-Fc format as described herein, the antibody has two binding sites for each antigen (FIG. 1A). However, in some embodiments a bispecific antibody may be a hybrid ABP, which may have a first binding region that is defined by a first light chain variable region and a first heavy chain variable region, and a second binding region that is defined by a second light chain variable region and a second heavy chain variable region. It is envisioned by the invention that one of these binding regions may be defined by a heavy/light chain pair. In the context of the present invention the bispecific binding molecule may have a second antigen binding site, defined by variable regions of a main chain and a smaller chain, and a first, different binding site defined by a variable region of a scFv fragment that is included in the main chain of the binding molecule.

Methods of making a bispecific ABP are known in the art, e.g. chemical conjugation of two different monoclonal antibodies or for example, also chemical conjugation of two antibody fragments, for example, of two Fab, or preferably scFv fragments. Alternatively, bispecific ABPs are made by quadroma technology, that is by fusion of the hybridomas producing the parental antibodies. Because of the random assortment of H and L chains, a potential mixture of ten different antibody structures is produced of which only one has the desired binding specificity.

The bispecific ABP of the invention can act as a monoclonal antibody (mAb) with respect to each target. In some embodiments the antibody is chimeric, humanized or fully human. A bispecific ABP may for example be a bispecific tandem scFv, a bispecific Fab2, or a bispecific diabody.

Hence, in accordance with the first and second context the first and/or the second binding domain of the bispecific molecule of the invention are derived from an antibody, and more preferably the first and/or second binding domain are single chain constructs, such as an antibody derived scFv construct. The most preferred bispecific format is therefore the scFv₂-Fc format shown in FIG. 1A. In that format the first and the second binding domain are linked to each other by a protein linker comprising one or more antibody-derived human constant domains, such as preferably of an IgG (such as IgG1 or IgG4), for example they are linked via human IgG1 or IgG4 derived hinge, CH2 and CH3.

In addition, or alternatively, a bispecific antibody, such as of the invention may also have an “Fc-attenuated” CH2 domain (that includes the hinge region). This “Fc-attenuation” is achieved by deleting and/ or substituting (mutating) at least one of selected amino acid residues in the CH2 domain that are able to mediate binding to an Fc-receptor. In illustrative embodiments, the at least one amino acid residue of the hinge region or the CH2 domain that is able to mediate binding to Fc receptors and that is lacking or mutated, is selected from the group consisting of sequence position 228, 230, 231, 232, 233, 234, 235, 236, 237, 238, 265, 297, 327, and 330 (numbering of sequence positions according to the EU-index). In an illustrative example, such an Fc-attenuated ABP may contain at least one mutation selected from the group consisting of a deletion of amino acid 228, a deletion of amino acid 229, a deletion of amino acid 230, a deletion of amino acid 231, a deletion of amino acid 232, a deletion of amino acid 233, a substitution Glu233→Pro, a substitution Leu234→Val, a deletion of amino acid 234, a substitution Leu235→Ala, a deletion of amino acid 235, a deletion of amino acid 236, a deletion of amino acid 237, a deletion of amino acid 238, a substitution Asp265→Gly, a substitution Asn297→Gln, a substitution Ala327→Gln, and a substitution Ala330→Ser (numbering of sequence positions according to the EU-index). In the case of bispecific antibodies that bind to specific types of immune cells such as NK, NKT or T cells, e.g. directing them against tumor cells, Fc-attenuation may be desired to prevent binding of the antibodies to other types of Fc-receptor carrying cells such as macrophages which may lead to undesirable off-target activation of such cells.

In some preferred embodiments, the antigenic target protein is selected from a protein expressed on cells associated with proliferative disorders or infectious diseases. In the case of tumor diseases as preferred proliferative disorders such antigenic proteins expressed on cells associated with the tumor disease are known as tumor-associated antigens or tumor-specific antigens. The person of skill is aware of a wide variety and selection of such antigenic proteins which may be used as a target for treating a particular tumor disease or an infectious disease. A list of exemplary antigens associated with tumor diseases is ErbB2 (HER2), CD19, CD20, GD2, PD-L1, EGFR, and EGFRvIII. Other exemplary antigens are BCMA, CD7, CD22, CD33, CD47, CD96, CD123, CD157, CD244, CLL1, FLT3/CD135, LeY, SLAMF7/CD319, and TIM3. Exemplary antigens associated with viral infections are antigens encoded by pathogenic organisms and particles, such as viral particles, and may include for example viral antigens of HIV, HPV, HBV, HCV, EBV, CMV, Influenza Virus, SARS-COV-1, or SARS-COV-2. Antigen binding fragments of antibodies specifically binding these antigens are also well known in the art.

The binding molecule of the invention, which is bispecific, has a first binding domain which is capable of binding to NKG2D, preferably in an NKG2D-ligand competitive manner, wherein the NKG2D ligand is for example MICA. Whether or not an NKG2D-specific binding domain does compete with a ligand of an NKG2D receptor for receptor binding may be tested, as an example, in accordance with an assay as described herein in example 2 and FIG. 8.

In preferred embodiments of the invention, the first binding site of the bispecific molecule comprises an antibody heavy chain and an antibody light chain variable domain, each derived from, and competitively binding to the same antigen such as, for example, the antibody KYK-2.0. Such antibody is disclosed for example in the international publication WO 2010/017103A2, which is included herein by reference in its entirety. Most preferably, the first binding site in accordance with the invention may comprise an NKG2D binding fragment as shown in SEQ ID NO: 2 or 4. Therefore, the first binding site may comprise the heavy chain and light chain CDR1-CDR3 sequences as comprised in the N-terminal scFv sequence as shown in SEQ ID NO: 2 or 4.

In other alternative or additional embodiments, the second binding site comprises an antibody heavy chain and an antibody light chain, each derived from, and competitively binding to the same antigen such as, for example, an antibody selected from FRP5, FMC63, Leu-16, ch14.18, atezolizumab, R1, cetuximab, or MR1-1.

In other preferred embodiments of the invention, the second binding domain may comprise an amino acid sequence which is or is derived from any of the following sequences:
SEQ ID NO: 5 (anti-CD19 scFv),
SEQ ID NO: 6 (anti-CD20 scFv),
SEQ ID NO: 7 (anti-EGFR scFv),
SEQ ID NO: 8 (anti-PD-L1 scFv),
   or wherein at least the second domain has an antigen binding domain similar to the antigen binding domain of any one of the scFv of SEQ ID NO: 5-8.

Most preferably the bispecific molecule of the invention comprises an amino acid sequence having at least 80%, 85%, 90%, 95%, 96%, 97%, 98%, most preferably 99% identity to the sequence shown in SEQ ID NO: 2 or 4, with the provision that the bispecific molecule of the invention comprises the CDR1 to CDR3 regions (heavy and light chain) of at least the NKG2D binding site shown in SEQ ID NO: 2 or 4, with not more than one amino acid addition, substitution, or deletion compared to these sequences. More preferably, the second binding site of the bispecific molecule of the invention comprises the CDR1 to CDR3 (heavy and light chain) of the second antigen binding domain specifically binding to ErbB2 in SEQ ID NO: 2 or 4, with not more than one amino acid addition, substitution, or deletion compared to these sequences. Most preferred is a bispecific molecule comprising the amino acid sequence of SEQ ID NO: 2 or 4.

It is well established that the cytokines IL-2 and IL-15 enhance the cytolytic activity of both NK cells and T cells. Both cytokines use a common βγ-receptor that is completed by a differing α-chain. The differential expression of the α-chain largely determines the biological activity of the cytokines. Both are capable of stimulating NK cells and T cells. However, whereas IL-2 stimulates T-regulatory cells (T regs), IL-15 appears to promote the expansion of e.g. CD8+ memory T cells and inhibit T regs (Ring et al. (2012) “Mechanistic and structural insight into the functional dichotomy between IL-2 and IL-15” Nat Immunol; 13:1187-1195; Waldmann (2006) “The biology of interleukin-2 and interleukin-15: implications for cancer therapy and vaccine design” Nat Rev Immunol; 6:595-601; Perna et al. (2013) “Interleukin 15 provides relief to CTLs from regulatory T cell-mediated inhibition: implications for adoptive T cell-based therapies for lymphoma” Clin Cancer Res; 19:106-117).

Hence, further preferred embodiments of the invention relate to the disclosed bispecific molecules which are further comprising an interleukin-15 domain fused to either the first and/or the second binding domain, wherein the interleukin-15 domain optionally is an interleukin-15 agonist. An IL-15 domain in context of the invention is preferably a polypeptide having an IL-15 amino acid sequence shown within the sequence of SEQ ID NO: 4.

In context of the herein disclosed invention the term “immune cell” shall refer to a cell type involved with a cell-mediated immune response, such as a cytotoxic immune response, in a mammal. Preferably, the immune cell is a cytotoxic cell, such as a cell expressing NKG2D protein and preferably is a T cell, NK cell, or NKT cell. In the context of the herein disclosed invention the immune cell is either an autologous immune cell or an allogeneic immune cell or immune cell line, and preferably is genetically engineered to have an increased expression of NKG2D.

Further, the immune cell may comprise a chimeric antigen receptor (CAR) comprising an extracellular NKG2D sequence, wherein the immune cell optionally further comprises an interleukin-15, or an interleukin-15 agonist as described herein. Preferably, an immune cell in accordance with the invention is an NK cell expressing an NKG2D CAR or an NK cell expressing NKG2D, and wherein the NK cell optionally further expresses an interleukin-15, or an interleukin-15 agonist.

The term “chimeric antigen receptor (CAR)” as used in the present invention refers to an artificial fusion protein comprising an extracellular domain capable of binding to an antigen, a transmembrane domain and an intracellular signaling domain which is usually composed of an immune costimulatory molecule and/or an intracellular signaling domain of an immune stimulatory protein. While the “extracellular domain capable of binding to an antigen” generally refers to any protein domain that mediates protein-protein interaction in an antigen specific manner, usually the term denotes antibody derived binding sites. In the present context however, the term denotes a protein domain which functions as a protein receptor such as the extracellular domain of NKG2D. The term “transmembrane domain” refers to a polypeptide derived from any membrane-binding protein or transmembrane protein, or a synthetic polypeptide mainly comprising hydrophobic residues such as leucine and valine. Such transmembrane domains may for example be derived from CD28 or CD3ζ. The term “intracellular signaling domain” refers to any oligopeptide or polypeptide known to function in a cell as transmitting a signal to cause activation or inhibition of a biological process. Such intracellular signaling domain is preferably derived from an immune stimulatory signaling protein of an immune cell, such as CD3, preferably the intracellular domain of CD3ζ. CARs according to the invention which comprise an extracellular domain of NKG2D are also referred to as chimeric NKG2D-based Activating Receptor (NKAR). A typical CAR will also include a signal peptide at its amino-terminal end, to direct the nascent translated protein into the endoplasmic reticulum so that the antigen binding domain will be presented on the surface of the immune cell in which the CAR is expressed. Where necessary the various specific domains of the CAR of the invention are connected by amino-acid linkers, which may be linkers as described above for the ABPs of the invention.

A preferred NKAR according to the invention comprises an extracellular protein sequence of NKG2D which comprises the epitope bound by the first binding domain.

Another additional or alternative embodiment of the invention further pertains to an NKAR as described wherein the NKAR in addition to an extracellular NKG2D sequence comprises:
(a) a hinge region such as a CD8α hinge region; and/or
(b) a transmembrane domain such as a transmembrane domain derived from CD3ζ or CD28; and
(c) an intracellular signalling domain such as an intracellular domain derived from CD3ζ; and optionally
(d) one or more intracellular costimulatory domains such as a CD28 derived intracellular domain.

Preferably either the transmembrane domain of CD3ζ or the transmembrane domain of CD28 is used.

The CAR of the invention is preferably provided already cell-surface expressed on modified immune cells such as NK, NKT or T cells, or as nucleic acid constructs comprising a nucleotide sequence encoding the CAR or NKAR of the invention. Preferably such nucleic acid constructs are transfectable into a target immune cell and are expressible for use in the various aspects and embodiments of the herein disclosed invention.

In an alternative variation of the first aspect, there is also provided a CAR or NKAR as described herein before, for use in the treatment of a disease is a subject, wherein the treatment comprises the administration of the CAR or NKAR, or alternatively of a nucleic acid construct encoding the CAR or NKAR, or a cell comprising the CAR or NKAR; and the additional administration of the binding molecule of the first or second aspect of the invention (in all disclosed variants or embodiments).

Preferred NKARs of the invention are shown in the disclosed examples 2 and 5.

In preferred embodiments of the invention an NKAR protein comprises the amino acid sequence shown in SEQ ID NO: 9 or 11, or an amino acid sequence at least 80%, 85%, 90%, 95%. 96%, 97%, 98%, 99% identical to the sequence shown in SEQ ID NO: 9 or 11.

In another preferred embodiment of the invention a nucleic acid construct (NAC) encoding an NKAR protein, and optionally an NKAR protein and an additional immune modulatory protein such as an IL-15 or an IL-15 together with an IL-15 receptor domain, or derivatives thereof. Preferably such an NAC encoding an NKAR comprises a nucleic acid sequence shown in SEQ ID NO: 10 or 12, or a nucleic acid sequence at least 80%, 85%, 90%, 95%. 96%, 97%, 98%, 99% identical to the sequence shown in SEQ ID NO: 10 or 12.

Embodiments disclosed herein with regard to nucleic acids and NACs as described herein under the fourth aspect of the invention equally apply to NACs encoding for the CAR or NKAR of the invention.

The use of IL-15 or an IL-15 agonist such as RD-IL15 is well known in the art. The amino acid sequence of RD-IL15 is provided herein in SEQ ID NO: 13, the encoding nucleic acid sequence is shown in SEQ ID NO: 14.

The molecules or combinations of the invention are preferably useful in the treatment of a proliferative disease, which preferably is selected from a cancer disease, such as a cancer, for example lung cancer, breast cancer, colorectal cancer, gastric cancer, liver cancer, pancreatic cancer, ovarian cancer, melanoma, kidney cancer, head and neck cancer, brain cancer, skin cancer, myeloma, lymphoma, or leukemia, preferably, wherein the proliferative disease is a cancer positive for an expression of the target antigenic protein.

In a third aspect, the invention pertains to an immune cell expressing an immune cell receptor, or an immune cell receptor, for use in the treatment of a disease in a subject, wherein the immune cell receptor is NKG2D, or a derivative thereof such as a CAR, and wherein the immune cell optionally further expresses an interleukin-15, or an interleukin-15 agonist, the treatment comprising an administration of a binding molecule recited in the first or second aspect, and an administration of the immune cell or the immune cell receptor to the subject.

In a fourth aspect, the invention pertains to an isolated nucleic acid, comprising one or more sequences encoding for a binding molecule recited in the first or second aspect of the invention. Preferably, the nucleic acid comprises a sequence as shown in SEQ ID NO: 3, or a sequence having at least 70, preferably 80, 85, 90, 95, 96, 97, 98, 99 or 100% sequence identity to the sequence shown in SEQ ID NO: 3.

For example, the component encoded by a nucleic acid of the invention may be all or part of one chain of an antibody of the invention; or the component may be a scFv of said binding molecule. The component encoded by such a nucleic acid may be all or part of one or another of the chains of an antibody of the invention; for example, the component encoded by such a nucleic acid may be a binding molecule of the invention. The nucleic acids of the invention may also encode a fragment, derivative, mutant, or variant of a binding molecule of the invention, and/or represent components that are polynucleotides suitable and/or sufficient for use as hybridization probes, polymerase chain reaction (PCR) primers or sequencing primers for identifying, analyzing, mutating or amplifying a polynucleotide encoding a polypeptide, anti-sense or inhibitory nucleic acids (such as RNAi/siRNA/shRNA or gRNA molecules) for inhibiting expression of a polynucleotide, and complementary sequences of the foregoing.

In particular embodiments of the invention, a nucleic acid of the invention comprises a nucleic acid having a sequence encoding a heavy or light chain CDR, a combination of heavy and/or light chain CDR1, CDR2 and CDR3 or a heavy or light chain variable domain, in each case as displayed in a sequence of an antibody of the invention (such as in SEQ ID NO: 2 preferably, or in any one of SEQ ID NO: 5, 6, 7, or 8), or a functional fragment thereof. In other embodiments, a nucleic acid of the invention comprises a nucleic acid sequence at least 60%, 65%, 70%, 75%, 80%, 85%, 90%; or 95% (preferably at least 75%) sequence identity to (or having no more than fifty, forty, thirty, twenty, fifteen, ten or five, preferably no more than three, two or one, base substitution(s), insertion(s) or deletion(s), to a sequence encoding any of the herein disclosed CDRs.

The nucleic acid according to the invention may be a DNA or RNA of genomic, mRNA, cDNA, or synthetic origin or some combination thereof, optionally linked to a polynucleotide to which it is not linked in nature. In some embodiments, such nucleic acid may comprise one or more (such as 2, 3, 4, 5, 6, 7, 8, 9, 10 or more than 20, in particular between 1 and about 5, or preferably all instances of a particular nucleotide in the sequence) unnatural (e.g. synthetic) nucleotides; and/or such nucleic acid may comprise (e.g. is conjugated to) another chemical moiety, such as a labeling group or an effector group; for example, a labeling group or an effector group as described elsewhere herein.

In one embodiment, the nucleic acid of the invention may be isolated or substantially pure. In another embodiment, the nucleic acid of the invention may be recombinant, synthetic and/or modified, or in any other way non-natural. For example, a nucleic acid of the invention may contain at least one nucleic acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human nucleic acid.

The nucleic acids can be any suitable length, such as about 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length. For example: siRNA nucleic acids may, preferably, be between about 15 to about 25 base pairs in length (preferably between about 19 and about 21 base pairs in length); an mRNA or DNA sequence encoding an ABP or a component thereof (such as a heavy or light chain or an IgG antibody) of the invention may, preferably, be between about 500 and 1,500 nucleotides. More preferably, a nucleic acid encoding a mammalian light chain of an antibody may be between about 630 and about 650 nucleotides, and one encoding a mammalian heavy chain of an antibody may be between about 1,300 and about 1,650 nucleotides. A nucleic acid can comprise one or more additional sequences, for example, regulatory sequences, and/or be part of a larger nucleic acid. The nucleic acids can be single-stranded or double-stranded and can comprise RNA and/or DNA nucleotides, and artificial variants thereof (e.g., peptide nucleic acids).

Changes can be introduced by mutation into the sequence of a nucleic acid of the invention. Such changes, depending on their nature and location in a codon, can lead to changes in the amino acid sequence of a polypeptide (e.g., an antigen binding protein) that it encodes. Mutations can be introduced using any technique known in the art.

In one embodiment, one or more particular amino acid residues may be changed using, for example, a site-directed mutagenesis protocol. In another embodiment, one or more randomly selected residues may be changed using, for example, a random mutagenesis protocol. However it is made, a mutant polypeptide can be expressed and screened for a desired property. Mutations can be introduced into a nucleic acid without significantly altering the biological activity of a polypeptide that it encodes. For example, one can make nucleotide substitutions leading to amino acid substitutions at non-essential amino acid residues.

Other changes that may be made (e.g. by mutation) to the sequence of a nucleic acid of the invention may not alter the amino acid sequence of the encoded polypeptide, but may lead to changes to its stability and/or effectiveness of expression of the encoded polypeptide. For example, by codon optimization, the expression of a given polypeptide sequence may be improved by utilizing the more common codons for a given amino acid that are found for the species in which the nucleotide is to be expressed. Methods of codon optimization, and alternative methods (such as optimization of CpG and G/C content), are described in, for example, Hass et al, 1996 (Current Biology 6:315); WO1996/09378; WO2006/015789 and WO 2002/098443.

In an alternative aspect, the invention pertains to a nucleic acid construct (NAC) comprising a nucleic acid of the fourth aspect and one or more additional sequence features permitting the expression of the encoded binding molecule (or further binding molecule), or a component of said binding molecule or further binding molecule (such as an antibody heavy chain or light chain) in a cell.

Such an NAC can comprise one or more additional features permitting the expression of the encoded binding molecule or component of said binding molecule (e.g. the antigen binding site) in a cell (such as in a host cell). Examples of NACs of the invention include, but are not limited to, plasmid vectors, viral vectors, mRNA, non-episomal mammalian vectors and expression vectors, for example, recombinant expression vectors. The nucleic acid constructs of the invention can comprise a nucleic acid of the invention in a form suitable for expression of the nucleic acid in a cell, such as a host cell, (see below). The nucleic acid constructs of the invention will be, typically, recombinant nucleic acids, and/or may be isolated and/or substantially pure. Recombinant nucleic acids will, typically, be non-natural; particularly if they comprise portions that are derived from different species and/or synthetic, in-vitro or mutagenic methods.

In some embodiments, an NAC of the invention comprises one or more constructs either of which includes a nucleic acid encoding either a heavy or a light antibody chain. In some embodiments, the NAC of the invention comprises two constructs, one of which includes a nucleic acid encoding the heavy antibody chain, the other of which includes a nucleic acid encoding the light antibody chain, such that expression from both constructs can generate a complete antibody molecule. In some embodiments, the NAC of the invention comprises a construct which includes nucleic acids encoding both heavy and light antibody chains, such that a complete antibody molecule can be expressed from one construct. In other embodiments, an NAC of the invention can comprise a single construct that encodes a single chain polypeptide which is sufficient to form an ABP of the invention; for example, if the encoded binding molecule is a scFv or a single-domain antibody (such as a camelid antibody).

In some embodiments, the NAC of the invention includes sequences encoding all or part of a constant region, enabling an entire, or a part of, a heavy and/or light chain to be expressed.

An NAC according to the invention may comprise (or consist of) an mRNA molecule which includes an open reading frame encoding a binding molecule of the invention, and for example together with upstream and downstream elements (such as 5′ and/or 3′ UTRs and/or a poly-A stretch) that enables expression of the binding molecule, and preferably enhancing stability of the mRNA and/or expression of the binding molecule. The use of mRNA as NACs to introduce into and express polynucleotides in cells is described, for example, in Zangi et al in Nat. Biotechnol. vol. 31, 898-907 (2013), Sahin et al (2014) Nature Reviews Drug Discovery 13:759 and by Thess et al in Mol. Ther. vol. 23 no. 9, 1456-1464 (2015). Particular UTRs that may be comprised in an mRNA NAC of the invention include: 5′ UTR of a TOP gene (WO2013/143699), and/or a histone stem-loop (WO 2013/120629). An mRNA NAC of the invention may further comprise one or more chemical modifications (EP 1 685 844); including a 5′-cap, such as m7G(5′)ppp, (5′(A,G(5′)ppp(5′)A or G(5′)ppp(5′)G and/or at least one nucleotide that is an analogue of naturally occurring nucleotides, such as phosphorothioates, phosphoroamidates, peptide nucleotides, methylphosphonates, 7-deaza-guanosine, 5-methylcytosine or inosine.

NACs, such as DNA-, retroviral- and mRNA-based NACs of the invention may be used in genetic therapeutic methods in order to treat or prevent diseases of the immune system (see Methods of Treatment below), whereby an NAC that comprises an expressible sequence encoding an ABP of the invention is administered to the cell or organism (e.g. by transfection). In particular, the use of mRNA therapeutics for the expression of antibodies is known from WO 2008/083949.

Preferably, in the context of the second and third aspect, the nucleic acid may comprise a sequence encoding for a protein having an amino acid sequence of any one of SEQ ID NO: 2 or 4, or a derivative of these sequences where the C-terminal anti-ErbB2 scFv sequence is replaced with any of the scFv sequences shown in SEQ ID NOs: 5-8.

In a fifth aspect, the invention pertains to a recombinant host cell comprising a nucleic acid or an NAC according to the above aspects. Preferably, such cell is capable of expressing the binding molecule (or component thereof) encoded by said NAC(s). For example, if a binding molecule of the invention comprises two separate polypeptide chains (e.g. a heavy and light chain of an IgG), then the cell of the invention may comprise a first NAC that encodes (and can express) the heavy chain of such binding molecule as well as a second NAC that encodes (and can express) the light chain of such binding molecule; alternatively, the cell may comprise a single NAC that encodes both chains of such binding molecule. In these ways, such a cell of the invention would be capable of expressing a functional (e.g. binding and/or inhibitory) binding molecule of the invention. A (host) cell of invention may be one of the mammalian, prokaryotic or eukaryotic host cells as described elsewhere herein, in particularly where the cell is a HEK293 cell or is a Chinese hamster ovary (CHO) cell.

In certain embodiments of such aspect, the (host) cell is a human cell; in particular, it may be a human cell that has been sampled from a specific individual (e.g. an autologous human cell). In such embodiments, such human cell can be propagated and/or manipulated in-vitro so as to introduce an NAC of the present invention. The utility of a manipulated human cell from a specific individual can be to produce a binding molecule of the invention, including to reintroduce a population of such manipulated human cells into a human subject, such as for use in therapy. In certain of such uses, the manipulated human cell may be introduced into the same human individual from which it was first sampled; for example, as an autologous human cell. Certain preferred immune cells are described elsewhere herein and preferably are selected from NK, NKT or T cells.

In a sixth aspect, the invention pertains to a pharmaceutical composition comprising: (i) a binding molecule of the first or second aspect, or (ii) a nucleic acid or NAC of the fourth aspect, or (iii) a recombinant host cell according to the third or fifth aspect, and a pharmaceutically acceptable carrier, stabilizer and/or excipient.

Preferably, the sixth aspect pertains to a pharmaceutical composition or package comprising:
(i) A binding molecule of the first or second aspect; or (ii) a nucleic acid, or (iii) a recombinant host cell of an aspect of the invention; and/or
an immune cell expressing the immune cell receptor, or the immune cell receptor, recited in the third aspect;
   together with a pharmaceutically acceptable carrier, stabilizer and/or excipient.

The pharmaceutical composition or package is to be used in therapy, the binding molecules, nucleic acids or NACs (or the cells, such as host cells) of the invention may be formulated into a pharmaceutical composition appropriate to facilitate administration to animals or humans. The term “pharmaceutical composition” means a mixture of substances including a therapeutically active substance (such as an ABP of the invention) for pharmaceutical use.

By way of example, the pharmaceutical composition of the invention may comprise between 0.1% and 100% (w/w) active ingredient, such as about 0.5%, 1%, 2%, 3%, 4%, 5%, 6%, 8% 10%, 15%, 20%, 25%, 30%, 40%, 50%, 60%, 70%, 80%, 90%, 95%, 96%, 97%, 98% or 99%, preferably between about 1% and about 20%, between about 10% and 50% or between about 40% and 90%.

As used herein the language “pharmaceutically acceptable” excipient, stabilizer or carrier is intended to include any and all solvents, solubilizes, fillers, stabilizers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of (or for use with) the invention is, typically, formulated to be compatible with its intended route of administration. Examples of routes of administration include oral, parenteral, e.g., intrathecal, intra-arterial, intravenous, intradermal, subcutaneous, transdermal (topical) and transmucosal administration.

In some embodiments, the pharmaceutical composition comprising a binding molecule or NAC is in unit dose form of between 10 and 1000 mg. In some embodiments, the pharmaceutical composition comprising a binding molecule or NAC is in unit dose form of between 10 and 200 mg. In some embodiments, the pharmaceutical composition comprising a binding molecule is in unit dose form of between 200 and 400 mg. In some embodiments, the pharmaceutical composition comprising a binding molecule or NAC is in unit dose form of between 400 and 600 mg. In some embodiments, the pharmaceutical composition comprising a binding molecule or NAC is in unit dose form of between 600 and 800 mg. In some embodiments, the pharmaceutical composition comprising a binding molecule or NAC is in unit dose form of between 800 and 1000 mg.

Exemplary unit dosage forms for pharmaceutical compositions comprising a binding molecule or NAC are tablets, capsules (e.g. as powder, granules, micro tablets or micro pellets), suspensions or as single-use pre-loaded syringes. In certain embodiments, kits are provided for producing a single-dose administration unit. The kit can contain both a first container having a dried active ingredient and a second container having an aqueous formulation. Alternatively, the kit can contain single and multi-chambered pre-loaded syringes.

Toxicity and therapeutic efficacy (e.g. effectiveness) of such active ingredients can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Active agents which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

In accordance with all aspects and embodiments of the medical uses and methods of treatment provided herein, the effective amount administered at least once to a subject in need of treatment with a binding molecule or NAC is, typically, between about 0.01 mg/kg and about 100 mg/kg per administration, such as between about 1 mg/kg and about 10 mg/kg per administration. In some embodiments, the effective amount administered at least once to said subject of an ABP or NAC is between about 0.01 mg/kg and about 0.1 mg/kg per administration, between about 0.1 mg/kg and about 1 mg/kg per administration, between about 1 mg/kg and about 5 mg/kg per administration, between about 5 mg/kg and about 10 mg/kg per administration, between about 10 mg/kg and about 50 mg/kg per administration, or between about 50 mg/kg and about 100 mg/kg per administration.

For the prevention or treatment of disease, the appropriate dosage of a binding molecule or NAC (or a pharmaceutical composition comprised thereof) will depend on the type of disease to be treated, the severity and course of the disease, whether the binding molecule or NAC and/or pharmaceutical composition is administered for preventive or therapeutic purposes, previous therapy, the patient's clinical history, age, size/weight and response to a binding molecule or NAC and/or pharmaceutical composition, and the discretion of the attending physician. The binding molecule or NAC and/or pharmaceutical composition is suitably administered to the patient at one time or over a series of treatments. If such binding molecule or NAC and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

In a seventh aspect, the invention pertains to a component for use in medicine, wherein the component is selected from the list consisting of: (i) a binding molecule or bispecific ABP of the first or second aspect, or (ii) an immune cell of the third aspect, (iv) a nucleic acid or NAC of the fourth aspect, or (v) a recombinant host cell according to the fifth aspect and (vi) a pharmaceutical composition or kit according to the sixth aspect.

In a related aspect, the invention also relates to a method of treating or preventing a disease, disorder or condition in a mammalian subject in need thereof, comprising administering to said subject at least once an effective amount of a modulating compound as desired above, or, and in particular administering to said subject at least once an effective amount of the binding molecule, the NAC, the (host) cells, or the pharmaceutical composition as described above.

In another related aspect, the invention also relates to the use of a product of the invention as describe above, or a modulating compound as described above (in particular a binding molecule of the invention) for the manufacture of a medicament, in particular for the treatment of a disease, disorder or condition in a mammalian subject, in particular where the disease, disorder or condition is one as set out herein.

Theterm “treatment” in the present invention is meant to include therapy, e.g. therapeutic treatment, as well as prophylactic or suppressive measures for a disease (or disorder or condition). Thus, for example, successful administration of a compound according to the invention prior to onset of the disease results in treatment of the disease. “Treatment” also encompasses administration of a compound of the invention after the appearance of the disease in order to ameliorate or eradicate the disease (or symptoms thereof). Administration of an NKG2D binding molecule of the invention after onset and after clinical symptoms, with possible abatement of clinical symptoms and perhaps amelioration of the disease, also comprises treatment of the disease. Those “in need of treatment” include subjects (such as a human subject) already having the disease, disorder or condition, as well as those prone to or suspected of having the disease, disorder or condition, including those in which the disease, disorder or condition is to be prevented.

In particular embodiments of these aspects, the modulating compound is one described above, and/or is a binding molecule, NAC, a (host) cell, or a pharmaceutical composition or kit of the present invention; in particular, is a binding molecule of the invention.

In other aspects described elsewhere herein, are provided methods to detect and/or diagnose a disease, disorder or condition in a mammalian subject.

In one particular embodiment, the disease, disorder or condition is characterized by a pathological immune response.

In another particular embodiment, the disease, disorder or condition is a proliferative disorder (or a condition associated with such disorder or disease), in particular when the product or modulating compound (such as a binding molecule, nucleic acid, NAC or recombinant host cell of the invention, in particular a binding molecule of the invention) pertains to inventive binding molecules comprising a binding site specific for a tumor antigen.

Another preferred disease, disorder or condition is an infectious disease, or an infectious state of a subject, in particular wherein the product or modulating compound (such as a binding molecule, nucleic acid, NAC or recombinant host cell of the invention, in particular a binding molecule of the invention) pertains to inventive binding molecules comprising a binding site specific for an antigen associated with the pathogenic organism or particle, such as preferably a viral particle.

Further, the invention is defined by the following itemized embodiments, which shall be understood in context of the above detailed description.
Item 1: A binding molecule which is at least bispecific comprising at least a first and a second binding domain for use in the treatment of a disease in a subject, wherein
   (a) the first binding domain is capable of binding to an epitope of NKG2-D type II integral membrane protein (NKG2D, SEQ ID NO: 1); and
   (b) the second binding domain is capable of binding to an epitope of an antigenic target protein expressed on or in a cell associated with the disease in the subject;
      wherein the treatment comprises an administration of the binding molecule to the subject and an administration of an immune cell expressing NKG2D to the subject.
Item 2: The binding molecule for use of item 1, wherein the first binding domain is capable of binding to NKG2D in an NKG2D-ligand competitive manner, wherein the NKG2D ligand is for example MHC class I polypeptide-related sequence A (MICA) or soluble MICA (sMICA).
Item 3: The binding molecule for use of item 1 or 2, wherein the first and/or the second binding domain are derived from an antibody, such as an antibody derived scFv construct.
Item 4: The binding molecule for use of any one of items 1 to 3, wherein the first and the second binding domain are linked to each other by a protein linker comprising one or more antibody-derived human constant domains, such as preferably of an IgG (such as IgG1 or IgG4), for example they are linked via human IgG1 or IgG4 derived hinge, CH2 and CH3.
Item 5: The binding molecule for use of any one of items 1 to 4, further comprising an interleukin-domain fused to either the first and/or the second binding domain, wherein the interleukin-15 domain optionally is an interleukin-15 agonist.
Item 6: The binding molecule for use of any one of items 1 to 5, wherein the immune cell is a cytotoxic cell, such as a cell expressing NKG2D and preferably is a T cell, natural killer (NK) cell, or NKT cell.
Item 7: The binding molecule for use of any one of items 1 to 6, wherein the immune cell comprises a chimeric antigen receptor (CAR) comprising an extracellular NKG2D sequence (NKAR), wherein the immune cell optionally further comprises an interleukin-15, or an interleukin-15 agonist.
Item 8: The binding molecule for use of item 7, wherein the NKAR further comprises:
   (a) a hinge region such as a CD8α hinge region; and/or
   (b) a transmembrane domain such as a transmembrane domain derived from CD3ζ or CD28; and
   (c) an intracellular signaling domain such as an intracellular domain derived from CD3ζ; and optionally
   (d) one or more intracellular costimulatory domains such as a CD28 derived intracellular domain.
Item 9: The binding molecule for use of any one of items 1 to 8, wherein the disease is a proliferative disease, preferably selected from a cancer disease, such as a cancer, for example lung cancer, breast cancer, colorectal cancer, gastric cancer, liver cancer, pancreatic cancer, ovarian cancer, melanoma, kidney cancer, head and neck cancer, brain cancer, skin cancer, myeloma, lymphoma, or leukemia, or wherein the disease is an infectious disease, such as a viral infection, for example an infection with a virus selected from HIV, HPV, HBV, HCV, EBV, CMV, Influenza Virus, SARS-COV-1, or SARS-COV-2, preferably, wherein the proliferative disease is a cancer positive for an expression of the antigenic target protein, or wherein the infectious disease is a viral infection positive for an expression of the antigenic target protein.
Item 10: A binding molecule which is at least bispecific comprising at least a first and a second binding domain, wherein
   (a) the first binding domain is capable of binding to an epitope of NKG2-D type II integral membrane protein (NKG2D, SEQ ID NO: 1); and
   (b) the second binding domain is capable of binding to an epitope of an antigenic target protein expressed on or in a cell associated with a disease in the subject;
      wherein the first and the second binding domain are antibody scFv constructs and are linked to each other via human IgG1 or IgG4 derived hinge, CH2 and CH3 domains.
Item 11: The binding molecule of item 10, further comprising an interleukin-15 domain fused to either the first and/or the second binding domain, wherein the interleukin-15 domain optionally is an interleukin-15 agonist.
Item 12: An immune cell expressing an immune cell receptor, or an immune cell receptor, for use in the treatment of a disease in a subject, wherein the immune cell receptor is NKG2D, or a derivative thereof such as a CAR (NKAR), and wherein the immune cell optionally further expresses an interleukin-15, or an interleukin-15 agonist, the treatment comprising an administration of the binding molecule recited in any one of items 1 to 11, and an administration of the immune cell or the immune cell receptor to the subject.
Item 13: The immune cell expressing the immune cell receptor, or the immune cell receptor, for use of item 12, wherein the immune cell is a cytotoxic cell, such as a cell expressing NKG2D and preferably is a T cell, natural killer (NK) cell, or NKT cell.
Item 14: The immune cell expressing the immune cell receptor, or the immune cell receptor, for use of item 12 or 13, wherein the immune cell is an autologous or allogeneic immune cell, and preferably is genetically engineered to have an increased expression of NKG2D.
Item 15: An isolated nucleic acid, comprising one or more sequences encoding for a binding molecule recited in any one of items 1 to 11.
Item 16: A recombinant host cell, comprising a nucleic acid recited in item 15.
Item 17: A pharmaceutical composition or package comprising:
   (a) (i) A binding molecule of any one of items 1 to 11; or (ii) an isolated nucleic acid recited in item 15, or (iii) a recombinant host cell of item 16; and/or
   (b) an immune cell expressing the immune cell receptor, or the immune cell receptor, recited in any one of items 12 to 14; or an expression construct for an NKAR as recited in item 7 or 8;
      together with a pharmaceutically acceptable carrier, stabiliser and/or excipient.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: shows the expression and purification of bispecific NKAB-ErbB2 antibody. (A) Schematic representation of bispecific antibody NKAB-ErbB2 consisting of an N-terminal NKG2D-specific scFv antibody fragment, hinge, CH₂ and CH₃ domains of human IgG₄, a (G₄S)₂ linker, and a C-terminal ErbB2-specific scFv antibody fragment. Disulfide bridges connecting the monomers within the homodimeric molecule are indicated by lines. (B) Analysis of elution fractions 1 and 2 after purification of NKAB-ErbB2 antibody via Protein-G affinity chromatography from culture supernatants of transiently transfected HEK 293T cells by SDS-PAGE under reducing conditions and Coomassie staining (left), and immunoblot analysis of purified NKAB-ErbB2 protein after SDS-PAGE under non-reducing (NR) or reducing conditions (R) with HRP-conjugated anti-human IgG antibody followed by chemiluminescent detection (right). The positions of NKAB-ErbB2 monomers and homodimers are indicated by black and gray arrowheads, respectively. (C) Binding of purified NKAB-ErbB2 to ErbB2 and NKG2D was investigated by flow cytometry with ErbB2-positive but NKG2D-negative MDA-MB453 breast carcinoma cells, ErbB2-negative but NKG2D-positive NK-92 NK cells, and double-negative MDA-MB468 breast carcinoma cells (dashed lines). Unstained cells (filled areas) and cells only incubated with secondary antibody (solid lines) were included as controls.

FIG. 2: shows the surface expression of NKG2D ligands (NKG2DL) by K562 erythroleukemia cells, MDA-MB453, MDA-MB468 and JIMT-1 breast carcinoma cells, and LNT-229 glioblastoma cells, determined by flow cytometry with BV786-conjugated anti-human MICA/B antibody, APC-conjugated anti-ULBP1 antibody, APC-conjugated anti-ULBP2/5/6 antibody, PE-conjugated anti-ULBP3 antibody, and PE-conjugated anti-ULBP4 antibody (left panels; solid lines). Cells treated with isotype antibodies served as controls (left panels; filled areas). Surface expression of ErbB2 by the same cancer cell lines was determined using APC-conjugated anti-ErbB2 antibody 24D2 (right panels; solid lines). Cells stained with an isotype antibody were included as controls (right panels; filled areas).

FIG. 3: shows the NKAB-ErbB2-mediated redirection of donor-derived lymphocytes to ErbB2-expressing cancer cells. (A) Proportions of NK (CD56+CD3−), NKT (CD56+ CD3+) and T cells (CD56− CD3+) in the peripheral blood mononuclear cells (PBMC) from healthy donors (D1-D3) used in (B) and (C). (B) Expression of NKG2D by NK, NKT and T cell populations of the cells shown in (A) gated according to their CD56 and CD3 expression was analyzed by flow cytometry with anti-NKG2D antibody (solid lines). An irrelevant antibody of the same isotype served as control (filled areas). (C) Cytotoxicity of the PBMCs shown in (A) against ErbB2-expressing MDA-MB453 breast carcinoma cells in the absence of bispecific antibody (black bars) or in the presence of increasing concentrations of recombinant NKAB-ErbB2 (white bars) was investigated in FACS-based cytotoxicity assays after co-incubation at an effector to target ratio (E/T) of 10:1 for 3 hours.

FIG. 4: shows the schematic representation of bispecific antibody NKAB-ErbB2 consisting of an N-terminal NKG2D-specific scFv antibody fragment, hinge, CH₂ and CH₃ domains of human IgG₄, a (G₄S)₂ linker, and a C-terminal scFv fragment derived from ErbB2-specific antibody FRP5 (A), the similar NKAB-ErbB2 (IgG₁) molecule carrying a human IgG₁ Fc region instead of IgG₄ (B), and the ErbB2-specific mini-antibody FRP5-Fc harboring an N-terminal scFv fragment derived from ErbB2-specific antibody FRP5, hinge, CH₂ and CH₃ domains of human IgG₁ (C). Disulfide bridges connecting the monomers within the homodimeric molecules are indicated by lines.

FIG. 5: shows the effect of NKAB-ErbB2 on the cell killing activity of donor-derived NK cells. (A) Expression of NKG2D and CD16 by the ex vivo expanded peripheral blood NK cells (pNK) from healthy donors (D4-D6) used in (B) was analyzed by flow cytometry with anti-NKG2D and anti-CD16 antibodies as indicated. (B) Cytotoxicity of the pNK cells shown in (A) against ErbB2-expressing MDA-MB453 breast carcinoma cells in the absence of bispecific antibody (black bars), or in the presence of increasing concentrations of recombinant NKAB-ErbB2 (white bars) or ErbB2-specific FRP5-Fc IgG₁ mini-antibody (gray bars) was investigated in FACS-based cytotoxicity assays after co-incubation at an effector to target ratio (E/T) of 5:1 for 3 hours.

FIG. 6: shows the redirection of donor-derived NK cells to ErbB2-expressing cancer cells by IgG₄- and IgG₁-based NKAB-ErbB2 antibodies. (A) Expression of NKG2D and CD16 by the ex vivo expanded peripheral blood NK cells (pNK) from healthy donors (D7-D9) used in (B) was analyzed by flow cytometry with anti-NKG2D and anti-CD16 antibodies as indicated. (B) Cytotoxicity of the pNK cells shown in (A) against ErbB2-expressing MDA-MB453 breast carcinoma cells in the absence of bispecific antibody (black bars), or in the presence of increasing concentrations of IgG₄-based NKAB-ErbB2 (white bars) or the similar IgG1-based NKAB-ErbB2 (IgG₁) molecule (gray bars) was investigated in FACS-based cytotoxicity assays after co-incubation at an effector to target ratio (E/T) of 5:1 for 3 hours.

FIG. 7: shows the generation of NKAR-NK-92 cells as an example for NKAR-NK cells. (A) Schematic representation of the lentiviral transfer plasmid encoding the NKG2D-based chimeric activating receptor NKAR under the control of the Spleen Focus Forming Virus promoter (SFFV). The receptor consists of an immunoglobulin heavy chain signal peptide (SP), the extracellular domain of NKG2D (amino acid residues 82-216), a flexible (G₄S)₂ linker (L), a Myc-tag (M), a CD8α hinge region (CD8α), and transmembrane and intracellular domains of CD3ζ. The NKAR sequence is followed by an internal ribosome entry site (IRES) and enhanced green fluorescent protein (EGFP) cDNA. (B) Expression of NKAR by sorted NKAR-NK-92 cells was analyzed by SDS-PAGE of whole cell lysate under non-reducing conditions and immunoblotting with CD3ζ-specific (left) and CD8a-specific antibodies (right), followed by HRP-conjugated secondary antibodies and chemiluminescent detection. Lysate of parental NK-92 cells was included as control. The positions of NKAR homodimers and monomers, CD3ζ homodimers, and NKAR-CD3ζ heterodimers are indicated by arrowheads. (C) Expression of the activating NK cell receptors NKG2D and NKAR, NKp30, NKp44, and NKp46 in sorted NKAR-NK-92 (dashed lines) and unmodified parental NK-92 cells (solid lines) was analyzed by flow cytometry using receptor-specific antibodies. NK-92 cells stained with irrelevant antibodies of the same isotype served as controls (filled areas). (D) Cytotoxicity of NKAR-NK-92 (filled circles) and parental NK-92 cells (filled triangles) against K562 erythroleukemia cells was investigated in FACS-based cytotoxicity assays after co-incubation at different effector to target ratios (E/T) for 3 hours. Mean values±SEM are shown; n=3. Data were analyzed by two-tailed unpaired Student's t-test. **, p<0.01; *, p<0.05.

FIG. 8: shows the restoration of sMICA-inhibited NKAR functionality by NKAB-ErbB2. (A) The ability of NKAB-ErbB2 to compete binding of soluble MICA to NKAR-NK-92 cells was determined by flow cytometry with APC-conjugated anti-His-tag antibody after incubation of cells with 2.5 μg/mL of His-tagged sMICA in the absence (solid line) or presence of 1.6 nM (0.25 μg/mL) or 16 nM (2.5 μg/mL) of NKAB-ErbB2 (dashed lines) as indicated. Cells treated only with secondary antibody served as control (filled area). (B) Inhibition of NKAR-NK-92 cell killing activity by 2.5 μg/mL of soluble MICA-Fc protein (sMICA) and restoration by addition of 0.16 nM (25 ng/mL) of NKAB-ErbB2 was investigated in FACS-based cytotoxicity assays after co-incubation with MDA-MB453 target cells at an E/T ratio of 5:1 for 3 hours. Recombinant human IgG₄ protein (25 ng/ml) served as isotype control. Mean values±SEM are shown; n=3. Data were analyzed by two-tailed paired Student's t-test. *, p<0.05; ns: not significant (p>0.05).

FIG. 9: shows the enhancement of NKAR-NK-92 cytotoxicity by NKAB-ErbB2. (A) The effect of NKAB-ErbB2 on specific cytotoxicity of NKAR-NK-92 cells against ErbB2-positive MDA-MB453 breast carcinoma cells was determined in FACS-based cytotoxicity assays after co-incubation at an effector to target ratio (E/T) of 5:1 for 3 hours in the absence or presence of increasing NKAB-ErbB2 concentrations (open bars). Parental NK-92 cells were included for comparison (filled bars). Mean values±SEM are shown; n=3. (B) Cytotoxicity of NKAR-NK-92 (filled circles) and NK-92 cells (filled triangles) in the absence, and NKAR-NK-92 (open circles) and NK-92 cells (open triangles) in the presence of 0.16 nM (25 ng/mL) of NKAB-ErbB2 against MDA-MB453, MDA-MB468 and JIMT-1 breast carcinoma cells and LNT-229 glioblastoma cells was investigated in FACS-based cytotoxicity assays after co-incubation at different E/T ratios for 3 hours. Mean values±SEM are shown; n=3. Data were analyzed by two-tailed unpaired Student's t-test (shown for NKAR-NK-92+NKAB-ErbB2 versus NKAR-NK-92). ***, p<0.001; **, p<0.01; *, p<0.05; ns: not significant (p>0.05).

FIG. 10: shows a comparative analysis of bispecific antibodies NKAB-ErbB2 and NKAB-ErbB2 (rev). (A) Schematic representation of NKAB-ErbB2 harboring an NKG2D-specific scFv fragment at the N-terminus, followed by hinge, CH₂ and CH₃ domains of human IgG₄, a (G₄S)₂ linker, and a C-terminal ErbB2-specific scFv fragment (left), and NKAB-ErbB2 (rev), in which the positions of NKG2D- and ErbB2-specific antibody domains are switched (right). (B) The effects of NKAB-ErbB2 (white bars) and NKAB-ErbB2 (rev) (gray bars) on specific cytotoxicity of NKAR-NK-92 (left) and parental NK-92 cells (right) against ErbB2-positive MDA-MB453 breast carcinoma cells was determined in FACS-based cytotoxicity assays after co-incubation at an effector to target ratio (E/T) of 5:1 for 3 hours in the absence (black bars) or presence of 0.32 nM (50 ng/mL) of bispecific antibodies. Mean values±SEM are shown; n=4.

FIG. 11: shows the activity of NKAB-ErbB2 and NKAR-NK-92 against ErbB2-expressing melanoma cells. (A) Cytotoxicity of NKAR-NK-92 in the absence (filled circles) or presence (open circles) of 0.16 nM (25 ng/ml) of NKAB-ErbB2 against murine B16-F10/ErbB2 melanoma cells expressing human ErbB2 was investigated in FACS-based cytotoxicity assays after co-incubation at different E/T ratios for 3 hours. Mean values+SEM are shown; n=3. (B) NKAR-NK-92 or parental NK-92 cells at a density of 5×105 cells/mL were incubated for 6 hours with B16-F10/ErbB2 cells at an E/T ratio of 1:1 in the absence (filled bars) or presence (open bars) of 0.16 nM (25 ng/mL) NKAB-ErbB2 as indicated. NK cells kept in the absence of tumor cells were included as controls. Supernatants were collected and the levels of IFN-γ, TNF-α, TNF-β, GM-CSF, RANTES (CCL5), MIP-1α (CCL3) and MIP-1β (CCL4) were measured using a cytometric bead array. Mean values±SEM are shown; n=3.

FIG. 12: shows the generation and functional characterization of NKAR-T cells. Peripheral blood mononuclear cells (PBMC) from a healthy donor were stimulated overnight with immobilized anti-CD3 and anti-CD28 antibodies. Activated PBMCs were then cultured for 24 hours in medium containing IL-7 and IL-15, before transduction with VSV-G pseudotyped NKAR-encoding lentiviral particles. (A) Four days later, transduction efficiency was determined by flow cytometric analysis of EGFP expression. T-cell purity and NKG2D surface expression were assessed using APC-conjugated anti-CD3 and PE-conjugated anti-NKG2D antibodies as indicated. Untransduced T cells were included as controls. (B) Expression of NKAR by transduced T cells was analyzed by SDS-PAGE of whole cell lysate under non-reducing conditions and immunoblotting with CD3ζ-specific antibody, followed by HRP-conjugated secondary antibody and chemiluminescent detection. Lysate of untransduced T cells was included as control. The positions of NKAR homodimers and monomers, CD3ζ homodimers, and NKAR-CD3ζ heterodimers are indicated by arrowheads. (C) Cytotoxicity of NKAR-T (filled circles) and untransduced T cells (filled triangles) in the absence, and NKAR-T (open circles) and untransduced T cells (open triangles) in the presence of 0.16 nM (25 ng/ml) of bispecific NKAB-ErbB2 antibody against ErbB2-positive MDA-MB453 breast carcinoma cells was investigated in FACS-based cytotoxicity assays after co-incubation at different E/T ratios for 3 hours.

FIG. 13: shows the combined in vivo antitumor activity of NKAR-NK-92 cells and NKAB-ErbB2 antibody against syngeneic glioblastoma in immunocompetent C57BL/6 mice. (A) Cytotoxicity of NKAR-NK-92 (filled circles) and NK-92 cells (filled triangles) in the absence, and NKAR-NK-92 (open circles) and NK-92 cells (open triangles) in the presence of 0.16 nM (25 ng/ml) of NKAB-ErbB2 against murine GL261/ErbB2 glioblastoma cells expressing human ErbB2 was investigated in FACS-based cytotoxicity assays after co-incubation at different E/T ratios for 3 hours. Mean values±SEM are shown; n=3. Data were analyzed by two-tailed unpaired Student's t-test (shown for NKAR-NK-92+NKAB-ErbB2 versus NKAR-NK-92). **, p<0.01. (B) GL261/ErbB2 cells (1×10⁶) were subcutaneously injected into the right flank of C57BL/6 mice. Seven days later, mice were treated by peritumoral injection of 1×10⁷ NKAR-NK-92 cells without (n=8) or with 5 μg of NKAB-ErbB2 antibody (n=9) admixed to the cells twice per week for 3 weeks. Control mice received parental NK-92 cells with NKAB-ErbB2 (n=9). Tumor growth in the individual animals was followed by caliper measurements. (C) Symptom-free survival of the mice. Data were analyzed by Kaplan-Meier plot and log-rank test. **, p<0.01; *, p<0.05; ns: not significant (p>0.05).

FIG. 14: shows bispecific NKAB antibodies targeting antigens other than ErbB2. (A) Schematic representation of bispecific NKAB antibodies consisting of an N-terminal NKG2D-specific scFv antibody fragment, hinge, CH₂ and CH₃ domains of human IgG₄, a (G₄S)₂ linker, and a C-terminal scFv antibody fragment targeting a tumor-associated antigen. scFv antibody fragments binding to EGFR, EGFRvIII, GD2, PD-L1, CD19 or CD20 are indicated as examples. Disulfide bridges connecting the monomers within the homodimeric molecules are indicated by lines. (B) Analysis of NKAB-CD19, NKAB-GD2 and NKAB-CD20 antibodies purified via Protein-G affinity chromatography from culture supernatants of transiently transfected HEK 293T cells by SDS-PAGE under non-reducing conditions and immunoblot analysis with HRP-conjugated anti-human IgG antibody followed by chemiluminescent detection. The positions of NKAB monomers and homodimers are indicated. (C) Binding of purified NKAB-CD19 to CD19, NKAB-CD20 to CD20 and NKAB-GD2 to GD2 was investigated by flow cytometry with tumor cells expressing the respective target antigens but negative for NKG2D (CD19- and CD20-positive Raji Burkitt lymphoma cells, or GD2-positive Mz-Mel-2 melanoma cells) as indicated (dashed lines). Binding to NKG2D was investigated using NKG2D-positive NK-92 cells which do not express the respective tumor antigens. Unstained cells (filled areas) and cells only incubated with secondary antibody (solid lines) were included as controls. (D) The effect of NKAB-CD19 and NKAB-CD20 on specific cytotoxicity of NKAR-NK-92 cells against CD19- and CD20-positive Raji Burkitt lymphoma cells was determined in FACS-based cytotoxicity assays after co-incubation at an effector to target ratio (E/T) of 5:1 for 3 hours in the absence or presence of increasing NKAB-CD19 (filled bars) or NKAB-CD20 concentrations (open bars).

FIG. 15: shows bispecific NKAB antibodies containing an additional IL-15 domain. (A) Schematic representation of bispecific NKAB/RD-IL15 antibodies consisting of an N-terminal IL-15 superagonist (RD-IL15), an NKG2D-specific scFv antibody fragment, hinge, CH₂ and CH₃ domains of human IgG₄, a (G₄S)₂ linker, and a C-terminal scFv antibody fragment targeting a tumor-associated antigen. scFv antibody fragments binding to ErbB2, EGFR, EGFRvIII, GD2, PD-L1, CD19 or CD20 are indicated as examples. Disulfide bridges connecting the monomers within the homodimeric molecules are indicated by lines (left panel). Bispecific NKAB/IL15 antibodies are similar to NKAB/RD-IL15 molecules but instead of the N-terminal IL-15 superagonist (RD-IL15) contain wildtype IL-15 (right panel). (B) Analysis of NKAB-CD19/RD-IL15, NKAB-GD2/RD-IL15, NKAB-CD20/RD-IL15 and NKAB-ErbB2/RD-IL15 antibodies purified via Protein-G affinity chromatography from culture supernatants of transiently transfected HEK 293T cells by SDS-PAGE under non-reducing conditions and immunoblot analysis with HRP-conjugated anti-human IgG antibody followed by chemiluminescent detection. The positions of NKAB/RD-IL15 monomers and homodimers are indicated. (C) Binding of purified NKAB-ErbB2/RD-IL15 to ErbB2, NKAB-CD19/RD-IL15 to CD19, NKAB-CD20/RD-IL15 to CD20 and NKAB-GD2/RD-IL15 to GD2 was investigated by flow cytometry with tumor cells expressing the respective target antigens but negative for NKG2D (ErbB2-positive MDA-MB453 breast carcinoma cells, CD19- and CD20-positive Raji Burkitt lymphoma cells, or GD2-positive Mz-Mel-2 melanoma cells) as indicated (dashed lines). Binding to NKG2D was investigated using NKG2D-positive NK-92 cells which do not express the respective tumor antigens. Unstained cells (filled areas) and cells only incubated with secondary antibody (solid lines) were included as controls. (D) The effect of NKAB-ErbB2/RD-IL15 on specific cytotoxicity of NKAR-NK-92 cells against ErbB2-positive MDA-MB453 breast carcinoma cells was determined in FACS-based cytotoxicity assays after co-incubation at an effector to target ratio (E/T) of 5:1 for 3 hours in the absence or presence of increasing NKAB-ErbB2/RD-IL15 concentrations (open bars). Parental NK-92 cells were included for comparison (filled bars). Mean values±SEM are shown; n=3.

FIG. 16: shows in (A) schematic representations of lentiviral transfer plasmids encoding under the control of the Spleen Focus Forming Virus promoter (SFFV) the NKG2D-based second-generation chimeric activating receptor NKAR(28.z) (upper panel), or the first-generation receptor NKAR together with IL-15 (middle panel) or the IL-15 superagonist RD-IL15 (bottom panel), with the NKAR and IL-15 sequences separated by a Porcine Teschovirus self-cleaving peptide (P2A). The receptor NKAR(28.z) consists of an immunoglobulin heavy chain signal peptide (SP), the extracellular domain of NKG2D (amino acid residues 82-216), a flexible (G₄S)₂ linker (L), a Myc-tag (M), a CD8α hinge region (CD8α), transmembrane and intracellular domains of CD28, and the intracellular domain of CD3ζ. NKAR(28.z), IL-15 and RD-IL15 sequences are followed by an internal ribosome entry site (IRES) and enhanced green fluorescent protein (EGFP) cDNA. (B) Cytotoxicity of NKAR(28.z)-NK-92 cells expressing the chimeric activating receptor NKAR(28.z) (filled circles) and parental NK-92 cells (filled triangles) in the absence, and NKAR(28.z)-NK-92 (open circles) and NK-92 cells (open triangles) in the presence of 0.16 nM (25 ng/ml) of NKAB-ErbB2 against ErbB2-positive MDA-MB453 breast carcinoma cells was investigated in FACS-based cytotoxicity assays after co-incubation at different E/T ratios for 3 hours. Mean values are shown; n=3. (C) Cytotoxicity of NKAR_RD-IL15-NK-92 cells expressing the chimeric activating receptor NKAR together with the IL-15 superagonist RD-IL15 (filled circles) and parental NK-92 cells (filled triangles) in the absence, and NKAR_RD-IL15-NK-92 (open circles) and NK-92 cells (open triangles) in the presence of 0.32 nM (50 ng/ml) of NKAB-ErbB2 against ErbB2-positive MDA-MB453 breast carcinoma cells was investigated in FACS-based cytotoxicity assays after co-incubation at different E/T ratios for 3 hours. Mean values±SEM are shown; n=3. (D) The effect of RD-IL15 provided to NKAR-expressing cells either in soluble form by recombinant NKAB-ErbB2/RD-IL15 protein or by direct co-expression in NKAR-expressing cells was investigated by culturing NKAR-NK-92 cells in regular growth medium with (filled black triangles) or without 100 IU/mL IL-2 (open triangles), or without IL-2 but in the presence of 20 ng/ml of NKAB-ErbB2/RD-IL15 (filled gray triangles), or by growing NKAR_RD-IL15-NK-92 cells with (filled black circles) or without 100 IU/mL IL-2 (open circles). Cell viability was analyzed by counting viable cells at the indicated time points using trypan blue exclusion. Mean values±SEM are shown; n=3.

FIG. 17: shows in (A) schematic representations of bispecific antibody NKAB-ErbB2 with intact intermolecular disulfide bridges within the IgG₄ hinge region (left) and modified monomeric NKAB-ErbB2 (C106S, C109S), wherein the cysteine residues within the hinge region are replaced by serine residues (right). (B) Cytotoxicity of NKAR-NK-92 cells in the absence of bispecific antibody (filled black bar), or in the presence of 25 ng/ml (0.16 nM) of homodimeric NKAB-ErbB2 (open bar) or 25 ng/ml (0.32 nM) of monomeric NKAB-ErbB2 (C106S, C109S) (filled gray bar) against murine GL261/ErbB2 glioblastoma cells expressing human ErbB2 was investigated in FACS-based cytotoxicity assays after co-incubation at an E/T ratio of 1:1 for 3 hours. Mean values±SEM are shown; n=3. Data were analyzed by two-tailed unpaired Student's t-test. **, p<0.01; *, p<0.05.

The sequences show:

### SEQ ID NO. 1 Shows the Amino Acid Sequence of Human NKG2D

```
                10 20 30 40
MGWIRGRRSR HSWEMSEFHN YNLDLKKSDF STRWQKQRCP
                50 60 70 80
VVKSKCRENA SPFFFCCFIA VAMGIRFIIM VAIWSAVFLN
                90 100 110 120
SLFNQEVQIP LTESYCGPCP KNWICYKNNC YQFFDESKNW
              130 140 150 160
YESQASCMSQ NASLLKVYSK EDQDLLKLVK SYHWMGLVHI
              170 180 190 200
PTNGSWQWED GSILSPNLLT IIEMQKGDCA LYASSFKGYI
              210
ENCSTPNTYI CMQRTV
```

### SEQ ID NO. 2 Shows the Amino Acid Sequence of the NKAB-ErbB2 Molecule (Complete Amino Acid Sequence)

MDWIWRILFLVGAATGAHSQVQLVESGGGLVKPGGSLRLSCAASGFTFS
SYGMHWVRQAPGKGLEWVAFIRYDGSNKYYADSVKGRFTISRDNSKNTL
YLQMNSLRAEDTAVYYCAKDRGLGDGTYFDYWGQGTTVTVSSGGGGSGG
GGSGGGGSQSALTQPASVSGSPGQSITISCSGSSSNIGNNAVNWYQQLP
GKAPKLLIYYDDLLPSGVSDRFSGSKSGTSAFLAISGLQSEDEADYYCA
AWDDSLNGPVFGGGTKLTVLASPPCPSCPAPEFLGGPSVFLFPPKPKDT
LMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLG
KGGGGSGGGGSQVQLQQSGPELKKPGETVKISCKASGYPFTNYGMNWVK
QAPGQGLKWMGWINTSTGESTFADDFKGRFDFSLETSANTAYLQINNLK
SEDSATYFCARWEVYHGYVPYWGQGTTVTVSSGGGGSGGGGSGGGGSDI
QLTQSHKFLSTSVGDRVSITCKASQDVYNAVAWYQQKPGQSPKLLIYSA
SSRYTGVPSRFTGSGSGPDFTFTISSVQAEDLAVYFCQQHFRTPFTFGS
GTKLEIK

### SEQ ID NO. 3 Shows the Nucleic Acid Sequence of the NKAB-ErbB2 Molecule (Complete Nucleic Acid Sequence)

ATGGACTGGATTTGGCGCATCCTGTTCCTCGTGGGAGCCGCCACCGGTGCCCATTCTCAGGTG
CAGCTGGTGGAATCTGGCGGCGGACTCGTGAAGCCTGGCGGCTCTCTGAGACTGAGCTGTGCC
GCCAGCGGCTTCACCTTCAGCAGCTACGGAATGCACTGGGTGCGCCAGGCCCCTGGCAAAGGA
CTGGAATGGGTGGCCTTCATCAGATACGACGGCAGCAACAAGTACTACGCCGACAGCGTGAAG
GGCCGGTTCACCATCTCCCGGGACAACAGCAAGAACACCCTGTACCTGCAGATGAACAGCCTG
CGGGCCGAGGACACCGCCGTGTACTACTGCGCCAAGGATAGAGGCCTGGGCGACGGCACCTA
CTTCGACTATTGGGGCCAGGGCACCACCGTGACCGTGTCTAGTGGCGGAGGCGGATCAGGCG
GCGGAGGATCAGGGGGAGGGGGATCTCAGTCTGCCCTGACACAGCCTGCCAGCGTGTCCGGA
TCTCCTGGCCAGAGCATCACCATCAGCTGCAGCGGCAGCAGCAGCAACATCGGCAACAACGCC
GTGAACTGGTATCAGCAGCTGCCCGGCAAGGCCCCCAAACTGCTGATCTACTACGACGACCTG
CTGCCCAGCGGCGTGTCCGATAGATTCAGCGGCTCCAAGAGCGGCACCAGCGCCTTTCTGGCC
ATCAGCGGCCTGCAGTCTGAGGACGAGGCCGACTACTATTGCGCCGCCTGGGACGACAGCCTG
AACGGCCCTGTGTTTGGAGGCGGCACCAAGCTGACAGTGCTGGCTAGCCCCCCATGCCCATCA
TGCCCAGCACCTGAGTTCCTGGGGGGACCATCAGTCTTCCTGTTCCCCCCAAAACCCAAGGACA
CTCTCATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAAGCCTC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGGCGGAGGCGGATCAGGCGGCGGAGGATCCCAGGT
GCAGCTGCAGCAGTCTGGCCCCGAGCTGAAGAAACCCGGCGAGACAGTGAAGATCTCCTGCAA
GGCCTCCGGCTACCCCTTCACCAACTACGGCATGAATTGGGTCAAGCAGGCCCCAGGCCAGGG
CCTGAAATGGATGGGCTGGATCAACACCAGCACCGGCGAGAGCACCTTCGCCGACGACTTCAA
GGGCAGATTCGACTTCAGCCTGGAAACCAGCGCCAACACCGCCTATCTGCAGATCAACAATCT
GAAGTCCGAGGACAGCGCTACCTACTTCTGCGCCAGATGGGAGGTGTACCACGGCTACGTGCC
ATACTGGGGACAGGGAACAACAGTGACAGTGTCCTCTGGCGGGGGAGGAAGTGGGGGGGGA
GGATCTGGGGGCGGAGGCAGTGATATCCAGCTGACCCAGAGCCACAAGTTTCTGAGCACCAGC
GTGGGCGACCGGGTGTCCATCACCTGTAAAGCCAGCCAGGACGTGTACAATGCCGTGGCTTGG
TATCAGCAGAAGCCTGGCCAGAGCCCTAAACTGCTGATCTATAGCGCCAGCAGCCGGTACACC
GGCGTGCCCTCTAGATTCACCGGATCTGGCAGCGGCCCTGACTTCACCTTTACCATCTCCAGCG
TGCAGGCCGAAGATCTGGCCGTGTATTTCTGCCAGCAGCACTTCCGGACCCCTTTCACCTTTGG
CTCCGGCACAAAGCTGGAAATCAAATGA

### SEQ ID NO. 4 Shows the Amino Acid Sequence of the NKAB-ErbB2_RD-IL15 Molecule (Complete Amino Acid Sequence)

MDWIWRILFLVGAATGAHSITCPPPMSVEHADIWVKSYSLYSRERYICN
SGFKRKAGTSSLTECVLNKATNVAHWTTPSLKCIRDPALVHQRPAPPGG
GSGGGGSGGGSGGGGSLQNWVNVISDLKKIEDLIQSMHIDATLYTESDV
HPSCKVTAMKCFLLELQVISLESGDASIHDTVENLIILANDSLSSNGNV
TESGCKECEELEEKNIKEFLQSFVHIVQMFINTSGGGSGGGSSGGGSQV
QLVESGGGLVKPGGSLRLSCAASGFTFSSYGMHWVRQAPGKGLEWVAFI
RYDGSNKYYADSVKGRFTISRDNSKNTLYLQMNSLRAEDTAVYYCAKDR
GLGDGTYFDYWGQGTTVTVSSGGGGSGGGGSGGGGSQSALTQPASVSGS
PGQSITISCSGSSSNIGNNAVNWYQQLPGKAPKLLIYYDDLLPSGVSDR
FSGSKSGTSAFLAISGLQSEDEADYYCAAWDDSLNGPVFGGGTKLTVLA
SPPCPSCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQ
EGNVFSCSVMHEALHNHYTQKSLSLSLGKGGGGSGGGGSQVQLQQSGPE
LKKPGETVKISCKASGYPFTNYGMNWVKQAPGQGLKWMGWINTSTGEST
FADDFKGRFDFSLETSANTAYLQINNLKSEDSATYFCARWEVYHGYVPY
WGQGTTVTVSSGGGGSGGGGSGGGGSDIQLTQSHKFLSTSVGDRVSITC
KASQDVYNAVAWYQQKPGQSPKLLIYSASSRYTGVPSRFTGSGSGPDFT
FTISSVQAEDLAVYFCQQHFRTPFTFGSGTKLEIK

### SEQ ID NO. 5 Shows the Amino Acid Sequence of an Anti-CD19 scFv in VH-Linker-VL Orientation (Complete Amino Acid Sequence)

EVQLQQSGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTTVTVSSGGGGSGGGGSGGGGSDIQMTQTTSSLS
ASLGDRVTISCRASQDISKYLNWYQQKPDGTVKLLIYHTSRLHSGVPSR
FSGSGSGTDYSLTISNLEQEDIATYFCQQGNTLPYTFGGGTKLEL

### SEQ ID NO. 6 Shows the Amino Acid Sequence of an Anti-CD20 scFv in VH-Linker-VL Orientation (Complete Amino Acid Sequence)

QVKLQESGAELVKPGASVKMSCKASGYTFTSYNMHWVKQTPGQGLEWIG
AIYPGNGDTSYNQKFKGKATLTADKSSSTAYMQLSSLTSEDSADYYCAR
SNYYGSSYWFFDVWGQGTTVTVSSGGGGSGGGGSGGGGSDIELTQSPTI
LSASPGEKVTMTCRASSSVNYMDWYQKKPGSSPKPWIYATSNLASGVPA
RFSGSGSGTSYSLTISRVEAEDAATYYCQQWSFNPPTFGGGTKLEIKRA

### SEQ ID NO. 7 Shows the Amino Acid Sequence of an Anti-EGFR scFv in VH-Linker-VL Orientation (Complete Amino Acid Sequence)

QVQLQESGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTTVTVSSGGGGSGGGGSGGGGSDIQLTQSPVILSV
SPGERVSFSCRASQSIGTNIHWYQQRTNGSPRLLIKYASESISGIPSRF
SGSGSGTDFTLSINSVESEDIADYYCQQNNNWPTTFGAGTKLEIK

### SEQ ID NO. 8 Shows the Amino Acid Sequence of an Anti-PD-L1 scFv in VH-Linker-VL Orientation (Complete Amino Acid Sequence)

EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSGGGGSGGGGSGGGGSDIQMTQSPSSLSAS
VGDRVTITCRASQDVSTAVAWYQQKPGKAPKLLIYSASFLYSGVPSRFS
GSGSGTDFTLTISSLQPEDFATYYCQQYLYHPATFGQGTKVEIK

### SEQ ID NO. 9 Shows the Amino Acid Sequence of an NKG2D-CAR (NKAR) (Complete Amino Acid Sequence)

MDWIWRILFLVGAATGAHSLFNQEVQIPLTESYCGPCPKNWICYKNNCY
QFFDESKNWYESQASCMSQNASLLKVYSKEDQDLLKLVKSYHWMGLVHI
PTNGSWQWEDGSILSPNLLTIIEMQKGDCALYASSFKGYIENCSTPNTY
ICMQRTVGGGGSGGGGSEQKLISEEDLALSNSIMYFSHFVPVFLPAKPT
TTPAPRPPTPAPTIASQPLSLRPEASRPAAGGAVHTRGLDPKLCYLLDG
ILFIYGVILTALFLRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPR

### SEQ ID NO. 10 Shows the Nucleic Acid Sequence of NKG2D-CAR (NKAR) (Complete Nucleic Acid Sequence)

ATGGATTGGATCTGGCGGATCCTGTTCCTCGTGGGAGCCGCCACAGGCG
CCCACAGCCTGTTCAATCAGGAAGTGCAGATCCCCCTGACCGAGAGCTA
CTGCGGCCCCTGCCCCAAGAACTGGATCTGCTACAAGAACAACTGCTAC
CAGTTCTTCGACGAGAGCAAGAATTGGTACGAGAGCCAGGCCAGCTGCA
TGAGCCAGAACGCCAGCCTGCTGAAGGTGTACAGCAAAGAGGACCAGGA
TCTGCTGAAGCTCGTGAAGTCCTACCACTGGATGGGCCTGGTGCACATC
CCCACCAATGGCAGCTGGCAGTGGGAGGACGGCAGCATCCTGAGCCCCA
ACCTGCTGACCATCATCGAGATGCAGAAGGGCGACTGCGCCCTGTACGC
CAGCAGCTTCAAGGGCTACATCGAGAACTGCAGCACCCCCAACACCTAC
ATCTGTATGCAGCGGACCGTGGGCGGAGGCGGAAGTGGCGGCGGAGGAT
CTGAGCAGAAGCTGATCTCCGAAGAGGACCTGGCCCTGAGCAACAGCAT
CATGTACTTCAGCCACTTCGTGCCCGTGTTTCTGCCCGCCAAGCCTACC
ACAACCCCAGCCCCTAGACCTCCTACACCCGCCCCTACAATCGCCAGCC
AGCCTCTGTCTCTGAGGCCCGAGGCTTCTAGACCTGCTGCAGGCGGAGC
TGTGCACACCAGGGGCCTGGACCCCAAGCTGTGCTACCTGCTGGACGGC
ATCCTGTTCATCTACGGCGTGATCCTGACCGCCCTGTTCCTGAGAGTGA
AGTTCAGCCGCAGCGCCGACGCCCCTGCCTACCAGCAGGGCCAGAACCA
GCTGTACAACGAGCTGAACCTGGGCAGGCGGGAGGAATACGACGTGCTG
GACAAGCGCAGAGGCCGGGACCCTGAGATGGGCGGCAAGCCCAGGCGGA
AGAACCCCCAGGAAGGCCTGTATAACGAACTGCAGAAAGACAAGATGGC
CGAGGCCTACAGCGAGATCGGCATGAAGGGCGAGCGGCGACGCGGCAAG
GGCCACGACGGCCTGTACCAGGGCCTGTCCACCGCCACCAAGGACACCT
ACGACGCCCTGCACATGCAGGCCCTGCCTCCCCGTTAA

### SEQ ID NO. 11 Shows the Amino Acid Sequence of an NKG2D-CAR with CD28 Costimulatory Sequence (NKAR(CD28z)) (Complete Amino Acid Sequence)

MDWIWRILFLVGAATGAHSLFNQEVQIPLTESYCGPCPKNWICYKNNCY
QFFDESKNWYESQASCMSQNASLLKVYSKEDQDLLKLVKSYHWMGLVHI
PTNGSWQWEDGSILSPNLLTIIEMQKGDCALYASSFKGYIENCSTPNTY
ICMQRTVGGGGSGGGGSEQKLISEEDLALSNSIMYFSHFVPVFLPAKPT
TTPAPRPPTPAPTIASQPLSLRPEASRPAAGGAVHTRGLDKPFWVLVVV
GGVLACYSLLVTVAFIIFWVRSKRSRLLHSDYMNMTPRRPGPTRKHYQP
YAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGH
DGLYQGLSTATKDTYDALHMQALPPR

### SEQ ID NO. 12 Shows the Nucleic Acid Sequence of an NKG2D-CAR with CD28 Costimulatory Sequence (NKAR(CD28z)) (Complete Nucleic Acid Sequence)

ATGGATTGGATCTGGCGGATCCTGTTCCTCGTGGGAGCCGCCACAGGCG
CCCACAGCCTGTTCAATCAGGAAGTGCAGATCCCCCTGACCGAGAGCTA
CTGCGGCCCCTGCCCCAAGAACTGGATCTGCTACAAGAACAACTGCTAC
CAGTTCTTCGACGAGAGCAAGAATTGGTACGAGAGCCAGGCCAGCTGCA
AAGCTCGTGAAGTCCTACCACTGGATGGGCCTGGTGCACATCCCCACCA
ATGAGCCAGAACGCCAGCCTGCTGAAGGTGTACAGCAAAGAGGACCAGG
ATCTGCTGTGGCAGCTGGCAGTGGGAGGACGGCAGCATCCTGAGCCCCA
ACCTGCTGACCATCATCGAGATGCAGAAGGGCGACTGCGCCCTGTACGC
CAGCAGCTTCAAGGGCTACATCGAGAACTGCAGCACCCCCAACACCTAC
ATCTGTATGCAGCGGACCGTGGGCGGAGGCGGAAGTGGCGGCGGAGGAT
CTGAGCAGAAGCTGATCTCCGAAGAGGACCTGGCCCTGAGCAACAGCAT
CATGTACTTCAGCCACTTCGTGCCCGTGTTTCTGCCCGCCAAGCCTACC
ACAACCCCAGCCCCTAGACCTCCTACACCCGCCCCTACAATCGCCAGCC
AGCCTCTGTCTCTGAGGCCCGAGGCTTCTAGACCTGCTGCAGGCGGAGC
TGTGCACACCAGGGGCCTGGACAAGCCCTTCTGGGTGCTGGTCGTGGTC
GGCGGAGTGCTGGCCTGTTACAGCCTGCTGGTCACCGTGGCCTTCATCA
TCTTTTGGGTCCGCAGCAAGCGGAGCCGGCTGCTGCACAGCGACTACAT
GAACATGACCCCAAGGCGGCCAGGCCCCACCCGGAAGCACTACCAGCCC
TATGCCCCTCCTAGGGACTTCGCCGCCTACCGGTCCAGAGTGAAGTTCA
GCCGCAGCGCCGACGCCCCTGCCTACCAGCAGGGCCAGAACCAGCTGTA
CAACGAGCTGAACCTGGGCAGGCGGGAGGAATACGACGTGCTGGACAAG
CGCAGAGGCCGGGACCCTGAGATGGGCGGCAAGCCCAGGCGGAAGAACC
CCCAGGAAGGCCTGTATAACGAACTGCAGAAAGACAAGATGGCCGAGGC
CTACAGCGAGATCGGCATGAAGGGCGAGCGGCGACGCGGCAAGGGCCAC
GACGGCCTGTACCAGGGCCTGTCCACCGCCACCAAGGACACCTACGACG
CCCTGCACATGCAGGCCCTGCCTCCCCGTTAA

### SEQ ID NO. 13 Shows the Amino Acid Sequence of an IL-15 Agonist (RD-IL-15) (Complete Amino Acid Sequence)

MDWIWRILFLVGAATGAHSITCPPPMSVEHADIWVKSYSLYSRERYICN
SGFKRKAGTSSLTECVLNKATNVAHWTTPSLKCIRDPALVHQRPAPPGG
GSGGGGSGGGSGGGGSLQNWVNVISDLKKIEDLIQSMHIDATLYTESDV
HPSCKVTAMKCFLLELQVISLESGDASIHDTVENLIILANDSLSSNGNV
TESGCKECEELEEKNIKEFLQSFVHIVQMFINTS

### SEQ ID NO. 14 Shows the Nucleic Acid Sequence of an IL-15 Agonist (RD-IL-15) (Complete Nucleic Acid Sequence)

ATGGACTGGATTTGGCGCATCCTGTTCCTCGTGGGAGCCGCCACCGGTG
CCCATTCTATCACCTGTCCTCCACCTATGAGCGTGGAACACGCCGACAT
CTGGGTCAAGAGCTACAGCCTGTACAGCAGAGAGCGGTACATCTGCAAC
AGCGGCTTCAAGAGAAAGGCCGGCACCAGCAGCCTGACCGAGTGTGTGC
TGAACAAGGCCACCAATGTAGCCCACTGGACCACACCTAGCCTGAAGTG
CATCAGAGATCCCGCTCTGGTGCATCAGCGACCTGCTCCACCTGGCGGA
GGATCTGGTGGTGGTGGAAGCGGAGGCGGATCTGGCGGCGGAGGTTCTC
TGCAGAATTGGGTCAACGTGATCTCCGACCTGAAGAAGATCGAGGACCT
GATCCAGAGCATGCACATCGACGCCACACTGTACACCGAGAGCGACGTG
CACCCTAGCTGTAAAGTGACCGCCATGAAGTGCTTTCTGCTGGAACTGC
AAGTGATCAGCCTGGAAAGCGGCGACGCCAGCATCCACGACACCGTGGA
AAACCTGATCATCCTGGCCAACGACAGCCTGAGCAGCAACGGCAATGTG
ACCGAGTCCGGCTGCAAAGAGTGCGAGGAACTGGAAGAGAAGAATATCA
AAGAGTTCCTGCAGAGCTTCGTGCACATCGTGCAGATGTTCATCAACAC
CAGCTGA

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

In general, the following experimental data were analyzed by two-tailed unpaired or paired Student's t-test and two-way ANOVA. Symptom-free survival was analyzed by Kaplan-Meier plot and log-rank (Mantel-Cox) test. P values<0.05 were considered statistically significant. Prism 8 software (GraphPad Software, La Jolla, CA) was used for all statistical calculations.

The examples show:

### Example 1: Design of Bispecific NKAB-ErbB2 Antibody

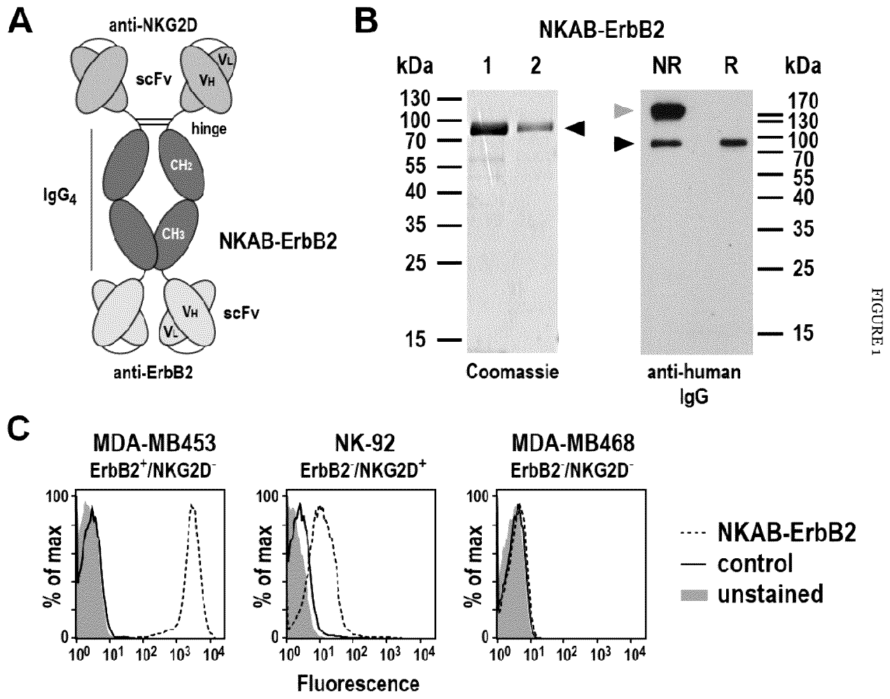

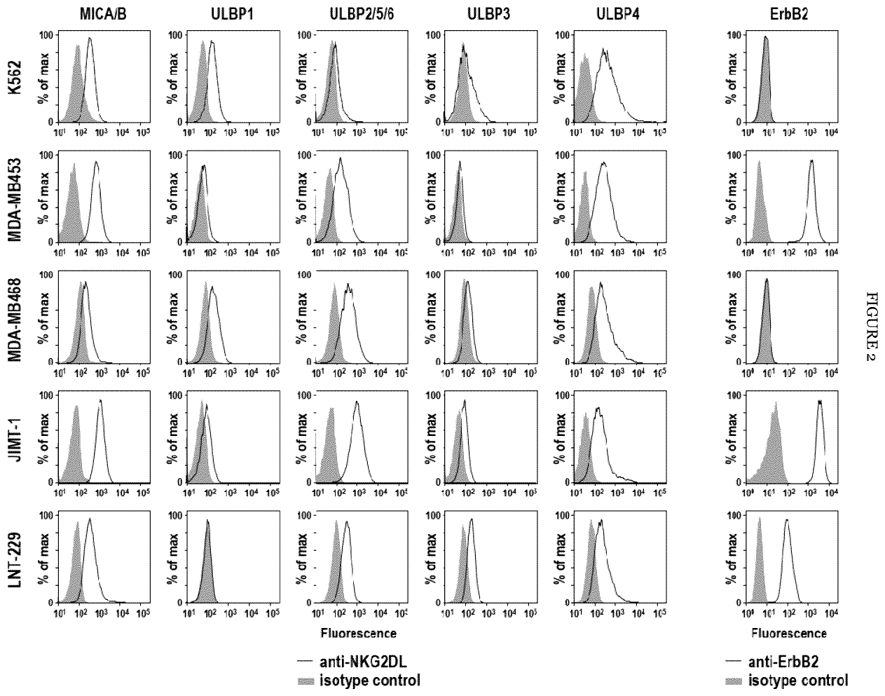

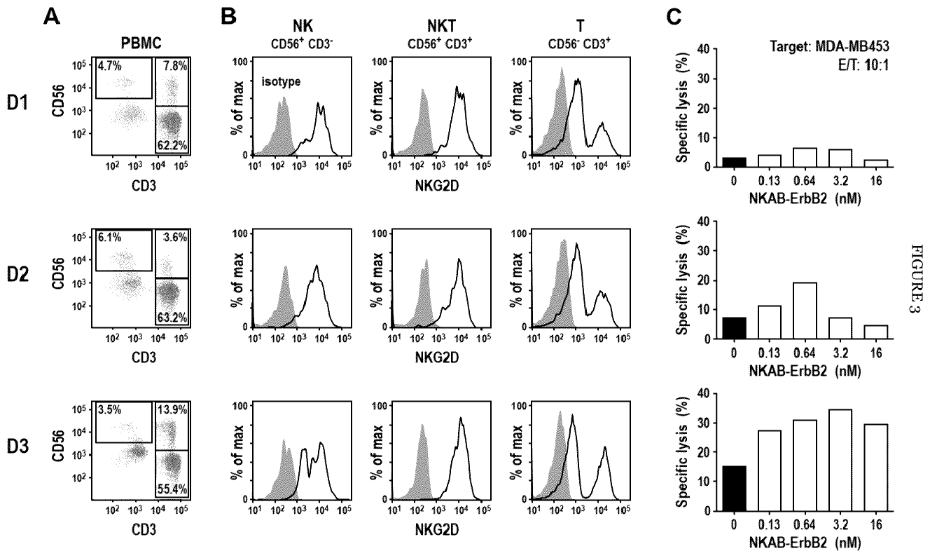

Generation of a bispecific antibody binding to NKGD2 and ErbB2: To target NKG2D-expressing lymphocytes to the tumor-associated antigen ErbB2, a bispecific antibody was designed that is similar in structure and molecular mass to an IgG molecule. This fusion protein (termed NKAB-ErbB2) carries an N-terminal single chain fragment variable (scFv) antibody domain derived from an NKG2D-specific antibody (Kwong, K. Y., Baskar, S., Zhang, H., Mackall, C. L. & Rader, C. Generation, affinity maturation, and characterization of a human anti-human NKG2D monoclonal antibody with dual antagonistic and agonistic activity. J Mol Biol 384, 1143-1156 (2008)) and a C-terminal second scFv domain derived from ErbB2-specific antibody FRP5 (Wels, W. et al. Construction, bacterial expression and characterization of a bifunctional single-chain antibody-phosphatase fusion protein targeted to the human erbB-2 receptor. Biotechnology (N Y) 10, 1128-1132 (1992)). To enable dimerization and to provide flexibility between the two scFv domains, they were linked via the hinge, CH₂ and CH₃ domains of human IgG₄, and a (G₄S)₂ peptide sequence (FIG. 1A). For expression as a secreted protein, the codon-optimized antibody sequence was fused in frame to an immunoglobulin heavy chain signal peptide sequence in a pcDNA3 expression plasmid. The recombinant NKAB-ErbB2 molecule was then expressed in transiently transfected HEK 293T cells and purified from culture supernatant by Protein G affinity chromatography. SDS-PAGE and immunoblot analysis under reducing and non-reducing conditions confirmed purity and identity of the protein, and revealed expression of the molecule as a tetravalent disulfide-linked homodimer, with only a minor fraction present in monomeric form under non-reducing conditions (FIG. 1B). Flow cytometric analysis demonstrated specific binding of purified NKAB antibody to ErbB2-expressing but NKG2D-negative MDA-MB453 breast carcinoma and ErbB2-negative but NKG2D-positive NK-92 cells, but not to MDA-MB468 breast carcinoma control cells which lack expression of ErbB2 and NKG2D (FIG. 1C; FIG. 2). This indicates that both, the NKG2D-specific and the ErbB2-specific binding domains in the molecule were functionally active.

### Example 2: Effect of Bispecific NKAB-ErbB2 Antibody

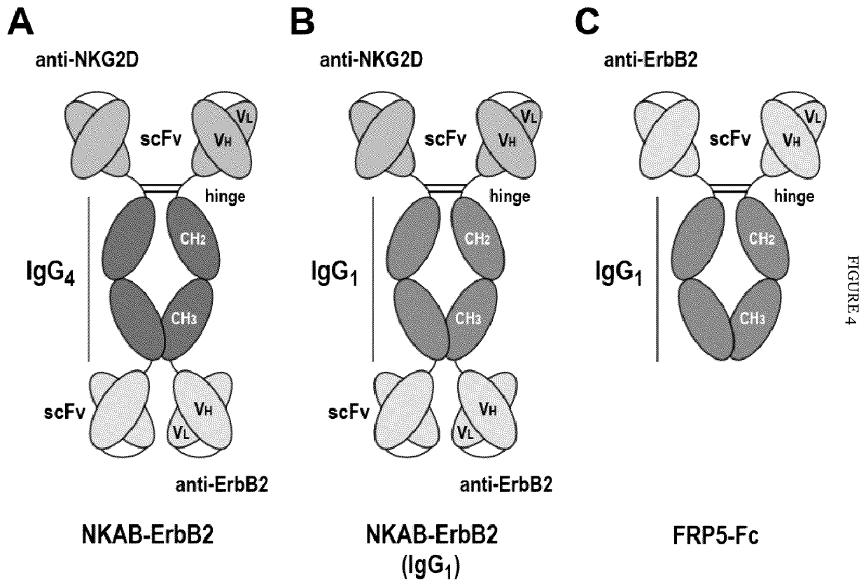

Effect of bispecific NKAB-ErbB2 antibody on the lytic activity of NKG2D-expressing peripheral blood lymphocytes and NK cells: Next it was investigated whether NKAB-ErbB2 influences the antitumor activity of NKG2D-positive peripheral blood lymphocytes in in vitro cytotoxicity assays using PBMCs from three healthy donors. Proportions of effector lymphocytes varied depending on the individual donor, with NK cells (CD56+ CD3−) ranging from 3.5 to 6.1%, NKT cells (CD56+ CD3+) from 3.6 to 13.9%, and T cells (CD56− CD3+) from 55.4 to 63.2% (FIG. 3A). While NK and NKT cells were largely NKG2D-positive, as expected only a smaller fraction of T cells expressed the receptor at high levels (FIG. 3B). In the absence of bispecific antibody, unstimulated PBMCs displayed little to moderate cytotoxicity against MDA-MB453 breast carcinoma cells ranging from 3.4 (D1) to 15.3% (D3) specific cell killing after 3 hours of co-incubation at an effector to target (E/T) ratio of 10:1 (FIG. 3C), likely due to NKG2D-mediated activation of the effector cells by the different NKG2D ligands endogenously expressed by the target cells (FIG. 2). In the presence of NKAB-ErbB2 antibody, cytotoxicity against the ErbB2-overexpressing cancer cells increased in a dose-dependent manner, with maximum cell killing of 2- to 2.6-fold over baseline reached at an antibody concentration of 0.64 nM (100 ng/ml; D1, D2) or 3.2 nM (500 ng/ml; D3). Cytotoxic activity decreased again at NKAB-ErbB2 concentrations above saturation of bispecific binding, indicative of competition by free antibody molecules.

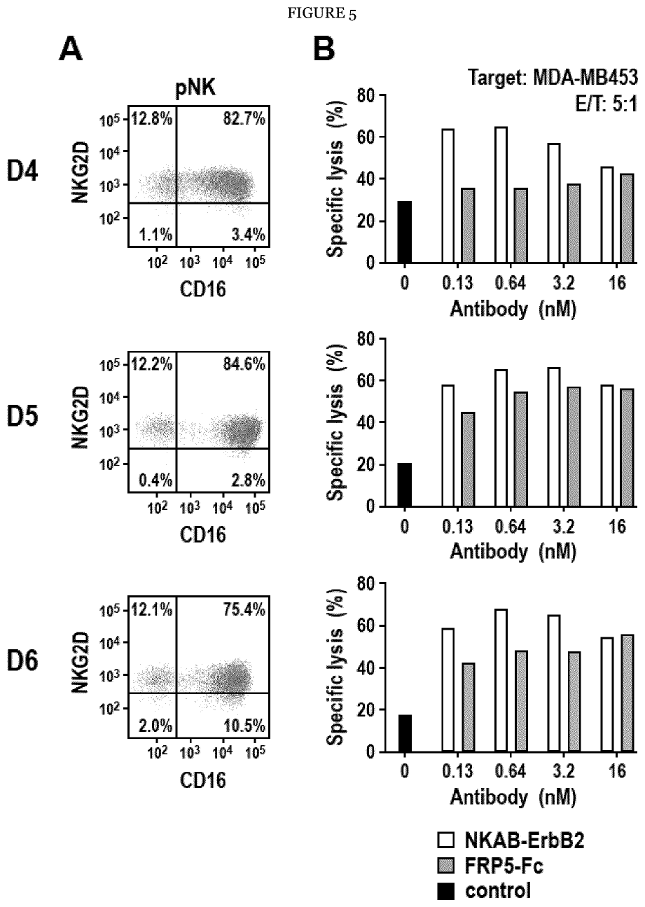

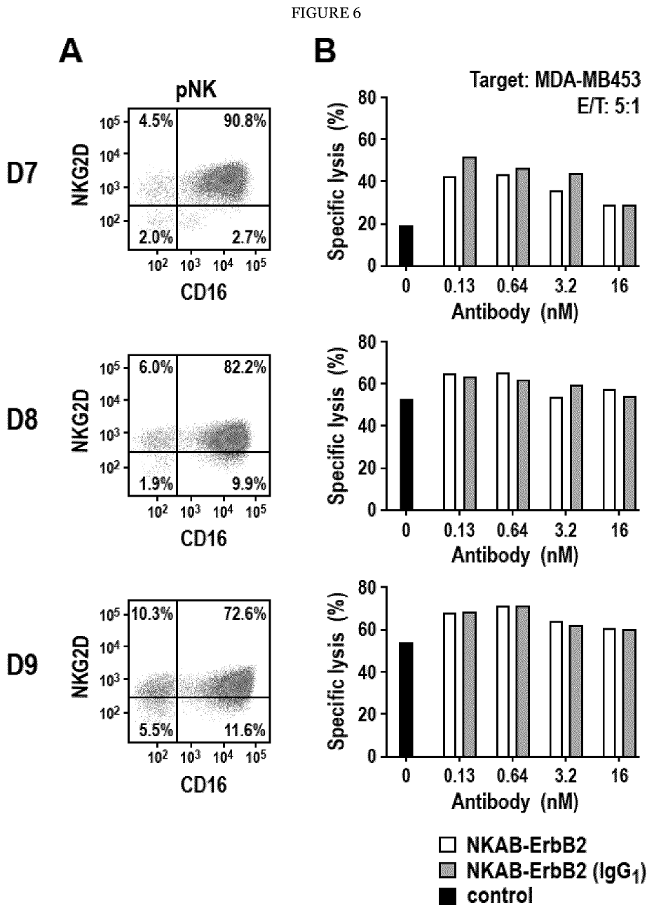

In a next set of experiments, instead of unstimulated donor-derived PBMCs the activity of the bispecific NKAB-ErbB2 molecule was evaluated with purified and ex vivo expanded peripheral blood NK (pNK) cells. To allow direct comparison between NKG2D- and CD16-mediated NK-cell cytotoxicity, an FRP5-Fc mini-antibody consisting of the same ErbB2-specific scFv domain used for NKAB-ErbB2 was included, but linked to the CD16-binding Fc portion of human IgG1 (FIG. 4). pNK cells from three different donors were expanded for two to three weeks in medium containing IL-2 and IL-15, with around 75 to 85% of cells in the resulting populations co-expressing NKG2D and CD16 (FIG. 5A). Upon co-incubation with MDA-MB453 tumor cells for 3 hours at an E/T ratio of 5:1, pNK cells from all donors demonstrated moderate baseline cytotoxicity in the absence of antibody, which was markedly enhanced in a concentration-dependent manner by NKAB-ErbB2, with maximum cell killing again reached at 0.64 nM (100 ng/ml; D4, D6) or 3.2 nM (500 ng/ml; D5). Also, antibody-dependent cell-mediated cytotoxicity (ADCC) triggered by FRP5-Fc through activation of CD16 resulted in increased antitumor activity of pNK cells, albeit for all donors to a lower degree than NKAB-ErbB2. To clearly distinguish NKG2D- and CD16-mediated effects, the NKAB-ErbB2 molecule was based on IgG₄, which cannot interact with CD16 with high affinity. Hence, to assess potential additive effects by ligating both, NKG2D and CD16 to ErbB2-positive tumor cells, also an NKAB-ErbB2 (IgG.) molecule was generated (FIG. 4) and its activity compared to that of the original IgG₄-based NKAB-ErbB2 antibody with pNK cells from another three donors. Thereby a very similar increase in cytotoxic activity of pNK cells against ErbB2-positive breast cancer cells was found for NKAB-ErbB2 and NKAB-ErbB2 (IgG₁) (FIG. 6).

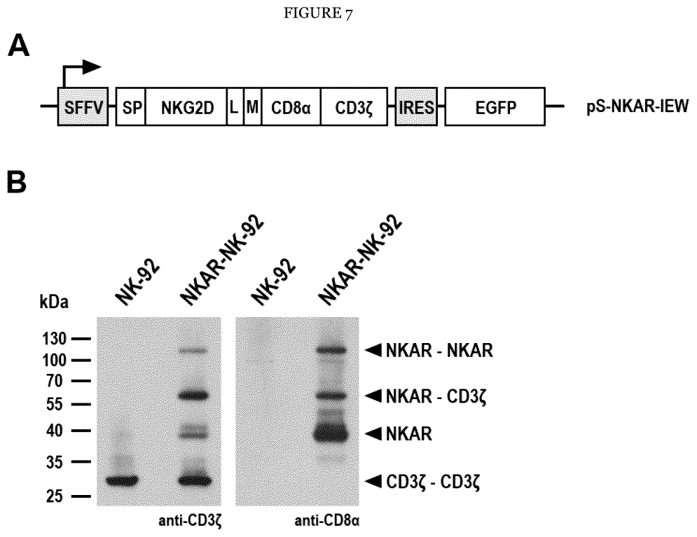

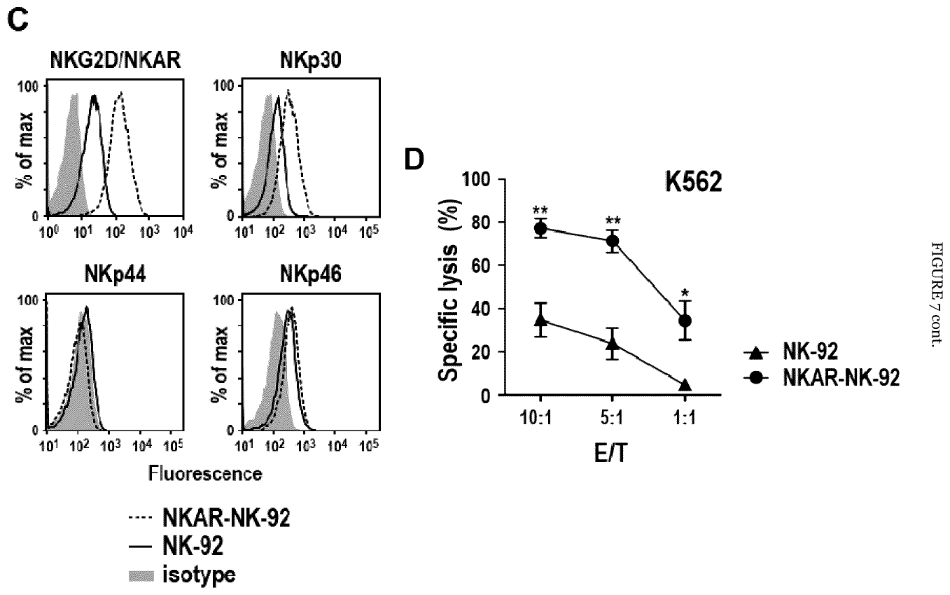

Enhancement of NK-cell activity by an NKG2D-based chimeric antigen receptor: Chimeric antigen receptors (CARs) which employ NKG2D for recognition of stress-induced NKG2D ligands can enhance cytotoxic activity of CAR-engineered T and NK cells towards tumor cells of various origins (Spear, P., Wu, M. R., Sentman, M. L. & Sentman, C. L. NKG2D ligands as therapeutic targets. Cancer Immun 13, 8 (2013) and Lazarova, M., Wels, W. S. & Steinle, A. Arming cytotoxic lymphocytes for cancer immunotherapy by means of the NKG2D/NKG2D-ligand system. Expert Opin Biol Ther (2020) and Obajdin, J., Davies, D. M. & Maher, J. Engineering of chimeric natural killer cell receptors to develop precision adoptive immunotherapies for cancer. Clin Exp Immunol (2020)). A similar CAR molecule (termed NKAR) was generated that harbors an immunoglobulin heavy chain signal peptide and the extracellular domain of human NKG2D, fused to transmembrane and intracellular domains of CD3ζ via a flexible linker, a Myc-tag and an optimized CD8α hinge region (Schönfeld, K. et al. Selective inhibition of tumor growth by clonal NK cells expressing an ErbB2/HER2-specific chimeric antigen receptor. Mol Ther 23, 330-338 (2015)). The CAR sequence was inserted into the self-inactivating lentiviral vector pSIEW, where it is encoded under the control of the Spleen Focus Forming Virus (SFFV) promoter and co-expressed with enhanced green fluorescent protein (EGFP) as a marker (FIG. 7A). VSV-G pseudotyped lentiviral vector particles were used for transduction of continuously expanding human NK-92 cells as a clinically relevant model for NK cells (Zhang, C. et al. Chimeric antigen receptor-engineered NK-92 cells: An off-the-shelf cellular therapeutic for targeted elimination of cancer cells and induction of protective antitumor immunity. Front Immunol 8, 533 (2017)), and resulting NKAR-NK-92 cells were enriched by flow cytometric cell sorting. NKAR expression was examined by SDS-PAGE under non-reducing conditions and immunoblot analysis with CD3ζ- and CD8α-specific antibodies, revealing the presence of NKAR monomers, disulfide-linked NKAR-NKAR homodimers and NKAR-CD3ζ heterodimers (FIG. 7B). Surface expression of the NKAR molecule was confirmed by flow cytometry, identified by a markedly increased NKG2D signal in NKAR-NK-92 cells when compared to parental NK-92 (FIG. 7C). Unexpectedly, NKAR expression also led to increased levels of NKp30, while NKp44 and NKp46 were not or only marginally affected (FIG. 7C). This may be due to a stabilizing effect of the CD3ζ-containing NKAR on overall CD3ζ levels and NKp30, which associates with CD3ζ for signaling (Pende, D. et al. Identification and molecular characterization of NKp30, a novel triggering receptor involved in natural cytotoxicity mediated by human natural killer cells. J Exp Med 190, 1505-1516 (1999)). NKAR expression resulted in strongly enhanced cytotoxicity of NKAR-NK-92 cells against K562 leukemia cells which express different NKG2D ligands (FIG. 7D; FIG. 2), indicating that the CAR molecule was functional.

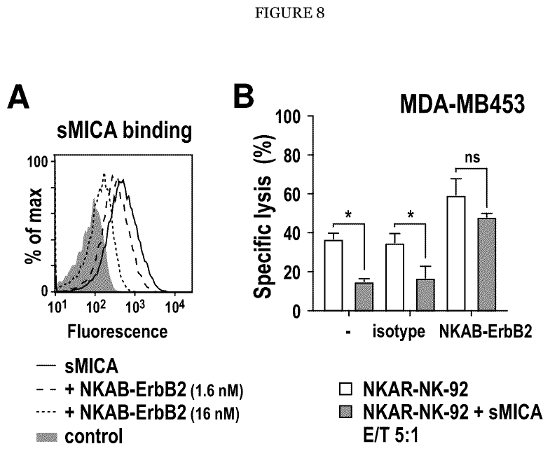

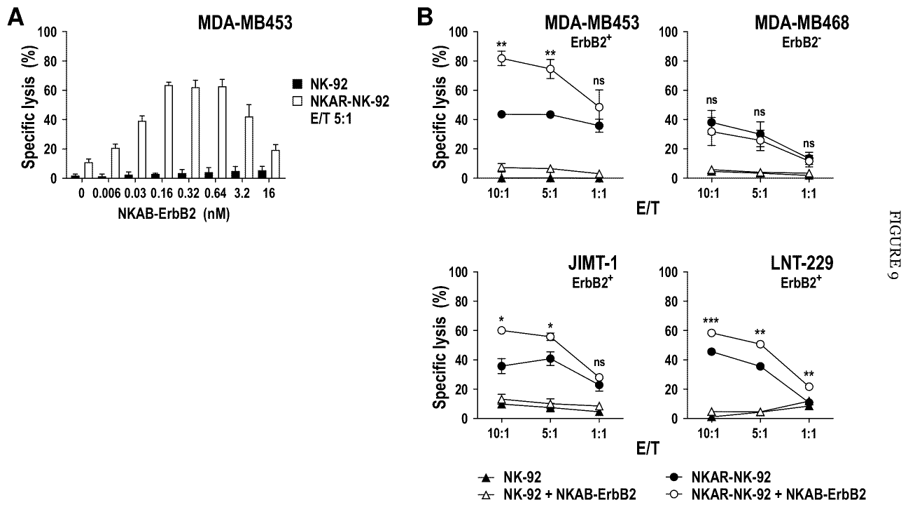

Restoration of sMICA-inhibited NKAR functionality by NKAB-ErbB2: Proteolytic shedding of NKG2D ligands such as MICA has been identified as a mechanism for cancer cells to evade NKG2D-mediated immune surveillance (Salih, H. R., Rammensee, H. G. & Steinle, A. Cutting edge: down-regulation of MICA on human tumors by proteolytic shedding. J Immunol 169, 4098-4102 (2002) and Lazarova, M., Wels, W. S. & Steinle, A. Arming cytotoxic lymphocytes for cancer immunotherapy by means of the NKG2D/NKG2D-ligand system. Expert Opin Biol Ther (2020)). To test whether this could also affect NK-92 cells expressing the NKG2D-based CAR, interaction of soluble MICA (sMICA) with NKAR-NK-92 cells was first investigated by flow cytometry. Thereby strong binding of sMICA to the surface of the CAR-NK cells was found, which was blocked in a concentration-dependent manner by NKAB-ErbB2 (FIG. 8A), indicating that the bispecific antibody can shield NKG2D. Occupation of the ligand binding site of the NKG2D-CAR by sMICA was also relevant for cytotoxic activity of NKAR-NK-92 cells, which was readily triggered by NKG2D ligands naturally expressed by MDA-MB453 breast cancer cells, but markedly inhibited in the presence of competing sMICA (FIG. 8B). Cell killing activity of the combination of NKAR-NK-92 cells and NKAB-ErbB2 was enhanced when compared to NKAR-NK-92 cells alone, but was not significantly affected by an excess of sMICA, suggesting that this strategy could overcome immune evasion due to ligand shedding.

### Example 3: Synergistic Effects of NKAB-ErbB2 and NKAR

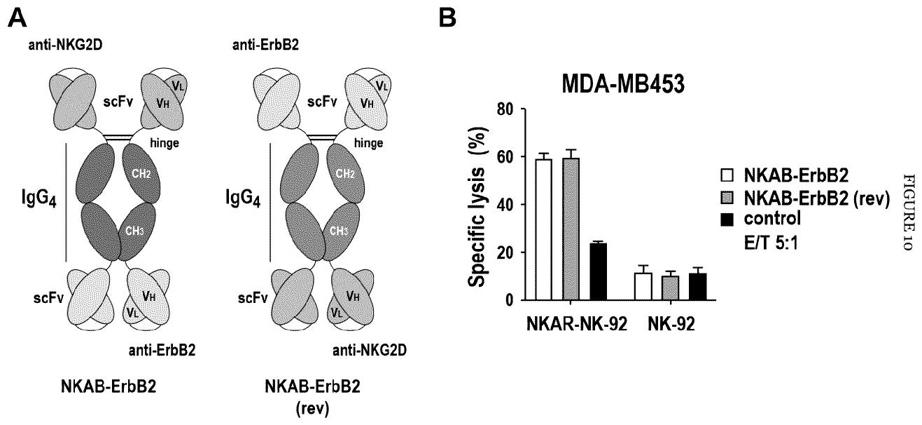

Next, the activity of NKAR-NK-92 cells in the absence or presence of NKAB-ErbB2 against MDA-MB453 breast cancer cells was investigated, which express high levels of ErbB2 and different NKG2D ligands (FIG. 2), but are largely resistant to parental NK-92 cells (Schönfeld, K. et al. Selective inhibition of tumor growth by clonal NK cells expressing an ErbB2/HER2-specific chimeric antigen receptor. Mol Ther 23, 330-338 (2015)). As seen with K562 target cells, NKAR-NK-92 cells already displayed increased lysis of MDA-MB453 cells in the absence of NKAB-ErbB2 (11.2% versus 2.1% of specific killing at an E/T ratio of 5:1), which was markedly enhanced to more than 60% specific lysis in the presence of 0.16 to 0.64 nM (25 to 100 ng/ml) of NKAB-ErbB2 (FIG. 9A). As observed with donor-derived PBMCs, cytotoxic activity of NKAR-NK-92 cells decreased again gradually at NKAB-ErbB2 concentrations above 0.64 nM, likely due to competition of productive cross-linking of effector and target cells by free antibody molecules. Addition of NKAB-ErbB2 also increased cytotoxicity of parental NK-92 cells against MDA-MB453 cells in this short-term assay, albeit not to meaningful levels (2.1% specific lysis in the absence versus a maximum of 5.7% in the presence of NKAB-ErbB2). This was likely due to the limited amount of endogenous NKG2D expressed by NK-92 (see FIG. 7C).

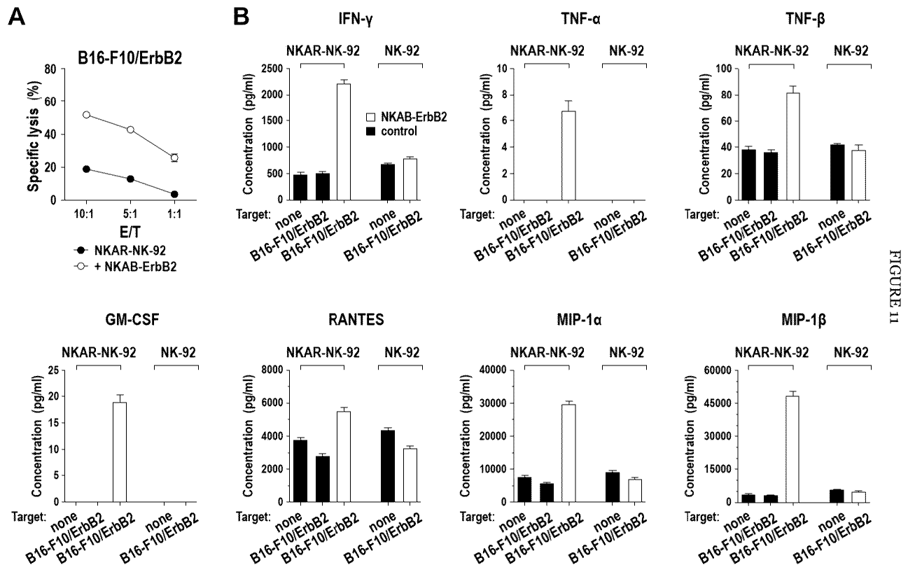

To test whether the combined effect of NKAB-ErbB2 and NKAR-expressing effector cells can consistently be achieved with ErbB2-positive cells of solid tumor origin, specific cytotoxicity of NKAR-NK-92 cells at increasing E/T ratios were tested with MDA-MB453 and JIMT-1 breast cancer cells, and LNT-229 glioblastoma cells in the absence or presence of 0.16 nM (25 ng/mL) NKAB-ErbB2. For comparison, MDA-MB468 breast cancer cells were included, which also harbor NKG2D ligands but are negative for ErbB2 (FIG. 2). Even at the highest E/T ratio applied, the four tested cell lines proved largely resistant to parental NK-92 cells, which was not changed significantly by the addition of NKAB-ErbB2 (FIG. 9B). In contrast, NKAR-NK-92 cells killed the NKG2D ligand positive targets with high efficiency, which in the case of the ErbB2-expressing tumor cells was further enhanced in a synergistic manner by NKAB-ErbB2. This included JIMT-1 breast cancer cells, which express NKG2D ligands and elevated levels of ErbB2 (FIG. 2), but are resistant to the clinically approved ErbB2-targeted therapeutics trastuzumab and lapatinib (O'Brien, N. A. et al. Activated phosphoinositide 3-kinase/AKT signaling confers resistance to trastuzumab but not lapatinib. Mol Cancer Ther 9, 1489-1502 (2010)). Importantly, while also displaying markedly enhanced sensitivity to NKAR-NK-92 cells, the addition of NKAB-ErbB2 did not further increase cytotoxicity of NKAR-NK-92 against ErbB2-negative MDA-MB468 cells, underscoring the specificity of the NKAB-ErbB2 effect (FIG. 9B). Demonstrating a large degree of flexibility of the NKAB protein design, the same synergy in the killing of MDA-MB453 cells seen with NKAR-NK-92 and the original NKAB-ErbB2 was observed when the CAR-NK cells were combined with recombinant NKAB-ErbB2 (rev), a molecule in which the positions of NKG2D- and ErbB2-specific binding domains relative to each other were exchanged (FIG. 10). In the case of ErbB2-expressing targets, NKAB-ErbB2-mediated activation of the NKG2D-CAR did not only trigger selective cytotoxicity, but also induced marked upregulation of pro-inflammatory cytokines such as IFN-γ, which is a hallmark of NK-cell activation (FIG. 11).

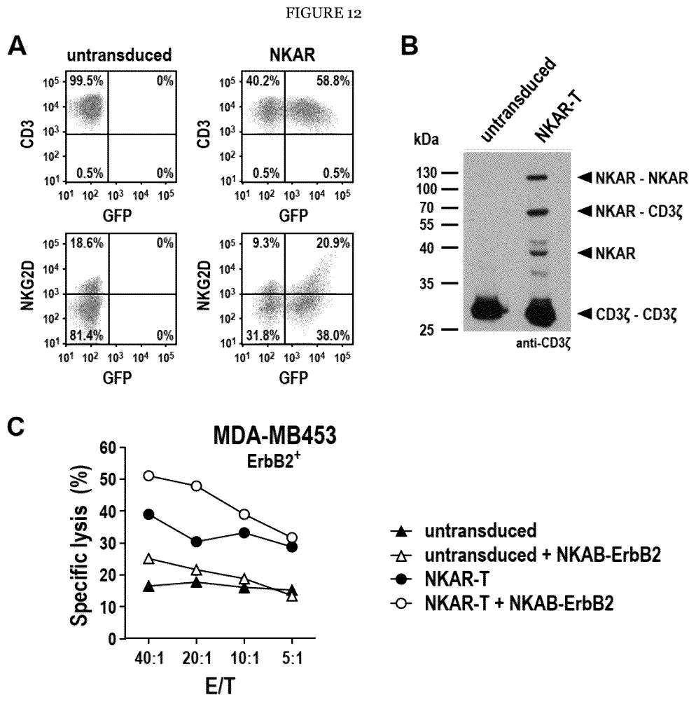

Initially, the concept of targeting tumor cells through an NKG2D-based CAR was developed using genetically engineered T cells (Zhang, T., Lemoi, B. A. & Sentman, C. L. Chimeric NK-receptor-bearing T cells mediate antitumor immunotherapy. Blood 106, 1544-1551 (2005)). To investigate whether the cytotoxicity of such CAR-T cells can be also enhanced by the NKAB-ErbB2 molecule, donor-derived T cells were transduced with the NKAR construct, and cytotoxicity of the resulting cell population against ErbB2-positive MDA-MB453 cells was tested in the absence or presence of 0.16 nM (25 ng/mL) NKAB-ErbB2. Thereby, similar to the findings according to the present invention with NK-92-derived NKAR-NK cells, the expression of NKAR on its own already increased cytotoxicity of NKAR-T cells, which was further enhanced by NKAB-ErbB2 (FIG. 12).

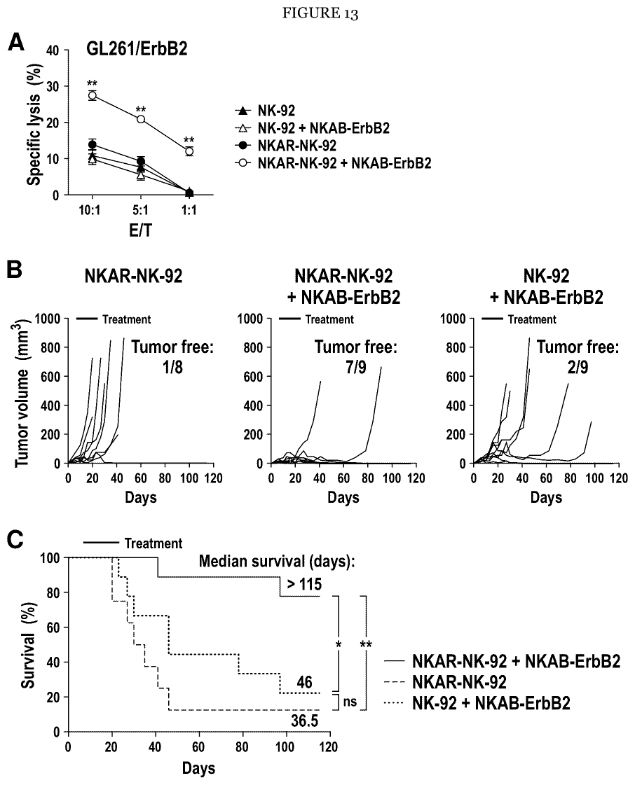

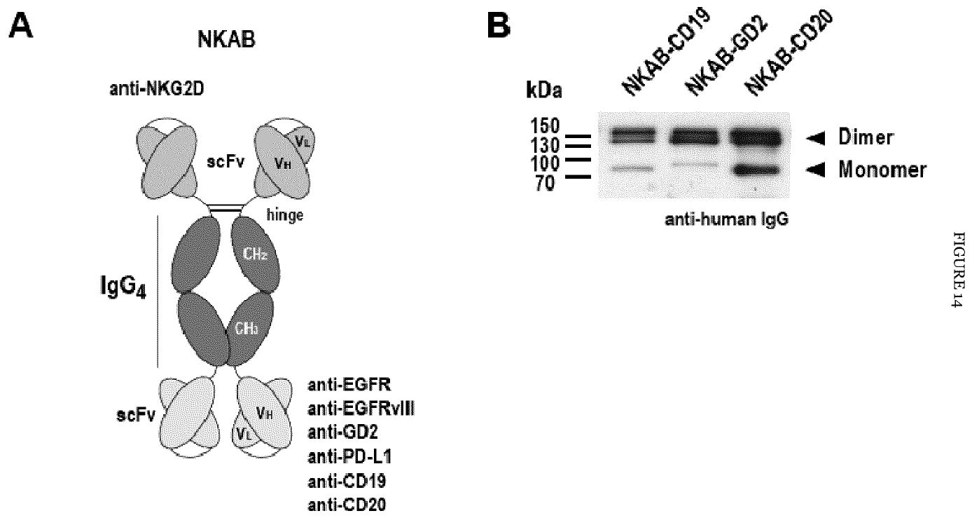

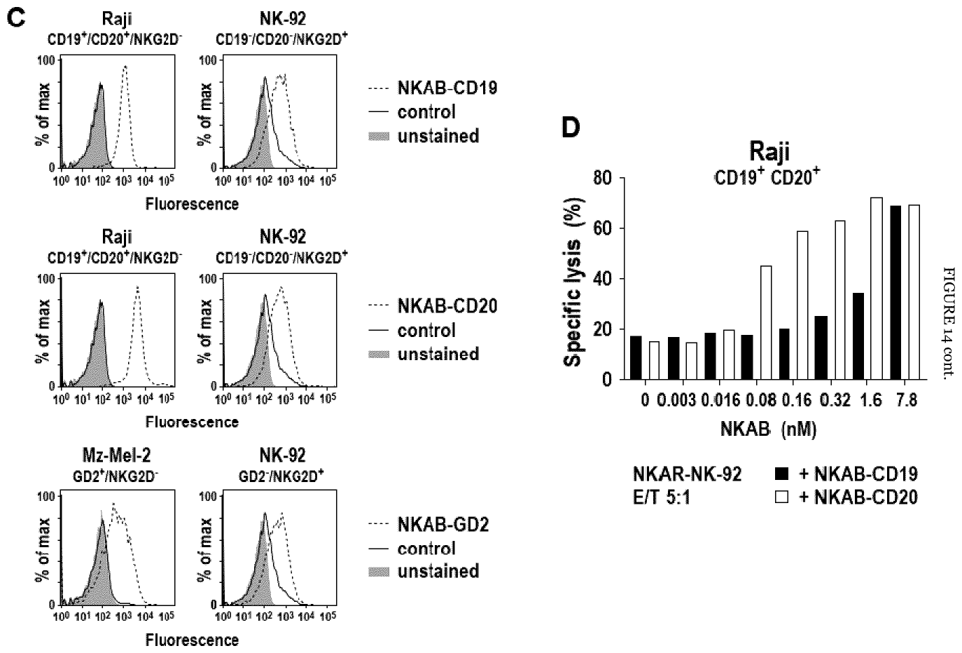

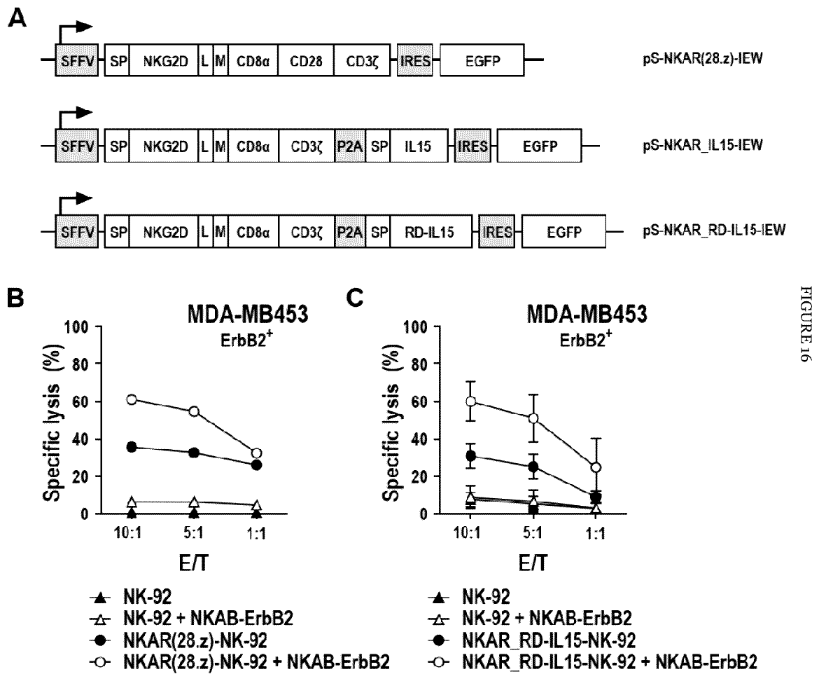

Durable responses upon treatment of ErbB2-positive glioblastoma tumors with NKAR-NK-92 cells and NKAB-ErbB2 antibody: To investigate the potential combined effect of NKAR-NK-92 cells and NKAB-ErbB2 antibody in a setting where tumor cells similar to cancer stem cells lack NKG2D ligands that could trigger the NKG2D-CAR directly (Paczulla, A. M. et al. Absence of NKG2D ligands defines leukaemia stem cells and mediates their immune evasion. Nature (2019)), a subcutaneous tumor model based on syngeneic GL261/ErbB2 glioblastoma tumors in immunocompetent C57BL/6 mice was established (Zhang, C. et al. Chimeric antigen receptor-engineered NK-92 cells: An off-the-shelf cellular therapeutic for targeted elimination of cancer cells and induction of protective antitumor immunity. Front Immunol 8, 533 (2017) and Zhang, C. et al. ErbB2/HER2-specific NK cells for targeted therapy of glioblastoma. J Natl Cancer Inst 108 (2016)). Seven days after tumor cell inoculation, mice were treated by peritumoral injection of 1×10⁷ NKAR-NK-92 or parental NK-92 cells with or without 5 μg of NKAB-ErbB2 antibody admixed to the injection medium. The treatment was repeated twice per week for three weeks. Since GL261/ErbB2 cells are of murine origin, endogenous NKG2D ligands expressed by these cells are not recognized by the NKAR molecule, which is based on human NKG2D. Consequently, in in vitro cytotoxicity assays no difference in sensitivity of GL261/ErbB2 cells to NKAR-NK-92 and parental NK-92 was found (FIG. 13A). For NK-92 cells this remained unchanged in the presence of NKAB-ErbB2. However, when NKAR-NK-92 cells were combined with NKAB-ErbB2, even at low E/T ratios a marked increase in cytotoxicity against GL261/ErbB2 was observed. Thereby, intact homodimeric NKAB-ErbB2 was more active against these target cells than a modified NKAB-ErbB2 (C106S, C109S) derivative which cannot form homodimers due to the lack of intermolecular disulfide bridges within the IgG₄ hinge region (FIG. 17). Likewise, NKAB-ErbB2-mediated recognition of murine melanoma cells genetically modified to express human ErbB2 induced cytokine secretion and specific lysis by NKAR-NK-92 cells (see FIG. 11A).

Also, in the in vivo setting, peritumoral treatment of established GL261/ErbB2 tumors with a combination of NKAR-NK-92 cells and NKAB-ErbB2 antibody twice weekly for three weeks was highly effective. Tumor outgrowth was controlled in eight out of nine animals in this group during therapy, and complete tumor regression was seen in seven of the mice thereafter, leaving no measurable tumors three months after the last treatment at termination of the experiment on day 115 (FIG. 13B). Since the NKAR molecule on its own cannot recognize GL261/ErbB2 cells, treatment with NKAR-NK-92 cells alone had no effect on tumor development during or after therapy, with only one out of eight mice in this group being tumor-free at endpoint analysis. This was most likely due to spontaneous rejection not related to the treatment, which was also previously observed in a small proportion of animals in this immunocompetent tumor model (Zhang, C. et al. ErbB2/HER2-specific NK cells for targeted therapy of glioblastoma. J Natl Cancer Inst 108 (2016)). While combination therapy with parental NK-92 cells and the NKAB-ErbB2 antibody showed some effect and resulted in delayed tumor growth, still only two of nine animals in this group presented as tumor-free on day 115 (FIG. 13B). The different kinetics in tumor growth among the treatment groups were also reflected in symptom-free survival. While mice in the NKAR-NK-92 only group had to be sacrificed due to disease progression earlier than in the group receiving the NK-92/NKAB-ErbB2 combination (median survival of 36.5 versus 46 days), this difference was not statistically significant (FIG. 13C). In contrast, with seven animals surviving and only two mice showing delayed tumor development, median survival in the NKAR-NK-92/NKAB-ErbB2 combination group was not reached in this experiment (>115 days).

### Example 5: Efficacy of Other Bispecific Antibodies than NKAB-ErbB2 Antibody

Bispecific antibodies binding to NKGD2 and target antigens other than ErbB2: To target NKG2D-expressing lymphocytes to surface antigens other than ErbB2, bispecific antibodies based on the structure of NKAB-ErbB2 were designed, but carrying instead of the ErbB2-specific scFv(FRP5) domain antibody fragments which recognize epidermal growth factor receptor (EGFR), the EGFR mutant form EGFRvIII, the disialoganglioside GD2, programmed death-ligand 1 (PD-L1), or the differentiation antigens CD19 or CD20 (FIG. 14A). The resulting NKAB molecules were expressed and purified as described above for NKAB-ErbB2. Immunoblot analysis under non-reducing conditions confirmed identity of the proteins and their ability to form disulfide-linked homodimers (shown for NKAB-GD2, NKAB-CD19 and NKAB-CD20 in FIG. 14B). Flow cytometric analysis revealed specific binding of the purified NKAB antibodies to NKG2D-positive NK-92 cells as well as tumor cells expressing the respective tumor-associated surface antigens, demonstrating that the molecules are functional and capable of bispecific binding (shown for NKAB-GD2, NKAB-CD19 and NKAB-CD20 in FIG. 14C). To test whether such NKAB antibodies can redirect the lytic activity of NKG2D-positive lymphocytes to tumor cells, the activity of NKAR-NK-92 cells was investigated in the absence or presence of NKAB-CD19 or NKAB-CD20 molecules against Raji Burkitt lymphoma cells which express both, CD19 and CD20. In the absence of NKAB molecules, NKAR-NK-92 cells displayed only limited lysis of Raji cells (up to 17.5% of specific killing at an E/T ratio of 5:1), which was markedly enhanced in a concentration-dependent manner up to 70% by NKAB-CD19 or NKAB-CD20 (FIG. 14D). These data demonstrate that NKAB molecules targeting different tumor-associated surface antigens can enhance and effectively redirect the cell killing activity of lymphocytes which express an NKG2D-based activating receptor.

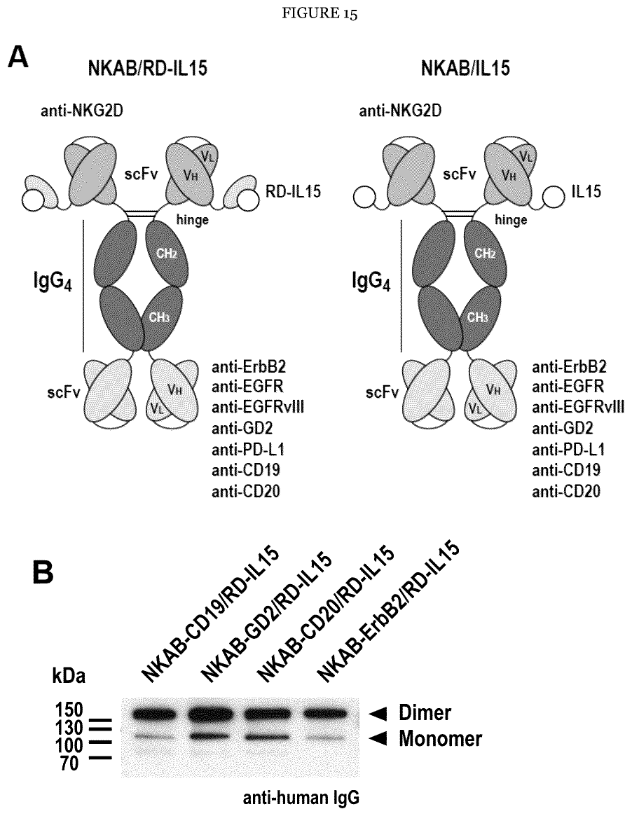

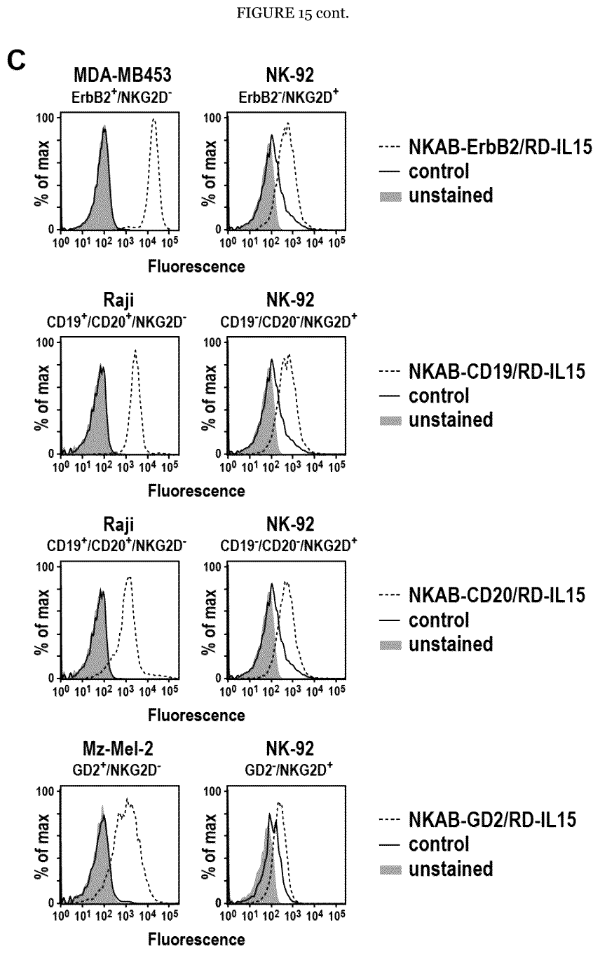

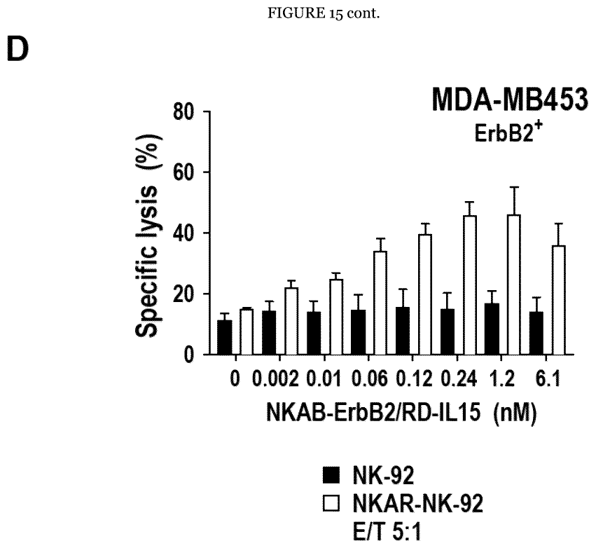

Bispecific NKAB antibodies containing an interleukin-15 domain: To provide NKAB antibodies with an additional IL-15 domain, codon-optimized sequences encoding the IL-15 superagonist RD-IL15 or wildtype IL-15 were inserted between the immunoglobulin heavy chain signal peptide and the target-cell specific N-terminal scFv antibody fragment of NKAB-ErbB2, NKAB-EGFR, NKAB-EGFRvIII, NKAB-GD2, NKAB-CD19 and NKAB-CD20 (FIG. 15A) (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012) and Zhu, X. et al. Novel human interleukin-15 agonists. J Immunol 183, 3598-3607 (2009)). The resulting NKAB/RD-IL15 and NKAB/IL15 molecules were expressed and purified as described above for NKAB-ErbB2. Immunoblot analysis under non-reducing conditions confirmed identity of the proteins and their ability to form disulfide-linked homodimers (shown for NKAB-ErbB2/RD-IL15, NKAB-GD2/RD-IL15, NKAB-CD19/RD-IL15 and NKAB-CD20/RD-IL15 in FIG. 15B). Flow cytometric analysis revealed specific binding of purified NKAB antibodies with IL-15 domains to NKG2D-positive NK-92 cells as well as tumor cells expressing the respective tumor-associated surface antigens, demonstrating that the molecules are functional, and like NKAB antibodies without IL-15, are capable of bispecific binding (shown for NKAB-ErbB2/RD-IL15, NKAB-GD2/RD-IL15, NKAB-CD19/RD-IL15 and NKAB-CD20/RD-IL15 in FIG. 15C).

To test whether such NKAB antibodies can redirect the lytic activity of NKG2D-positive lymphocytes to tumor cells, the activity of NKAR-NK-92 cells was investigated in the absence or presence of increasing concentrations of NKAB-ErbB2/RD-IL15 against MDA-MB453 breast cancer cells, which express high levels of ErbB2. Thereby, cell killing activity of NKAR-NK-92 cells was markedly increased in the presence of 0.002 to 6.1 nM of NKAB-ErbB2/RD-IL15 when compared to NKAR-NK-92 cells in the absence of antibody, with maximum lysis of 46% achieved at concentrations of 0.24 and 1.2 nM (FIG. 15D). Addition of NKAB-ErbB2/RD-IL15 also slightly increased cytotoxicity of parental NK-92 cells against MDA-MB453 cells (11.4% specific lysis in the absence versus a maximum of 16.7% in the presence of NKAB-ErbB2/RD-IL15). These data demonstrate that NKAB molecules containing an additional IL-15 domain are functionally active and like bispecific NKAB antibodies without IL-15 can enhance and effectively redirect the cell killing activity of lymphocytes which express an NKG2D-based activating receptor. Furthermore, NKAB antibodies with IL-15 domains also support the survival and growth of immune effector cells (shown for NKAB-ErbB2/RD-IL15 in FIG. 16D).

Combination of bispecific NKAB antibodies with lymphocytes expressing an NKG2D-based second-generation chimeric activating receptor: Inclusion of one or more costimulatory protein domains in chimeric antigen receptors in addition to CD3ζ can be beneficial with respect to the functionality of resulting gene-modified lymphocytes (Sadelain, M., Brentjens, R. & Rivière, I. The promise and potential pitfalls of chimeric antigen receptors. Curr Opin Immunol 21, 215-223 (2009)). To test whether in addition to the chimeric activating receptor NKAR with a single CD3ζ signaling domain also other CAR formats are functional in combination with a bispecific NKAB molecule, as another example a CAR molecule termed NKAR(28.z) was generated, that harbors an immunoglobulin heavy chain signal peptide and the extracellular domain of human NKG2D, fused via a flexible linker, a Myc-tag and an optimized CD8α hinge region to transmembrane and intracellular domains of CD28, and the intracellular domain of CD3ζ (Schönfeld, K. et al. Selective inhibition of tumor growth by clonal NK cells expressing an ErbB2/HER2-specific chimeric antigen receptor. Mol Ther 23, 330-338 (2015)). The CAR sequence was inserted into the self-inactivating lentiviral vector pSIEW, where it is encoded under the control of the Spleen Focus Forming Virus (SFFV) promoter and co-expressed with enhanced green fluorescent protein (EGFP) as a marker (FIG. 16A, upper panel). VSV-G pseudotyped lentiviral vector particles were used for transduction of continuously expanding human NK-92 cells as a clinically relevant model for NK cells (Zhang, C. et al. Chimeric antigen receptor-engineered NK-92 cells: An off-the-shelf cellular therapeutic for targeted elimination of cancer cells and induction of protective antitumor immunity. Front Immunol 8, 533 (2017)), and resulting NKAR(28.z)-NK-92 cells were enriched by flow cytometric cell sorting as described above for the initial NKAR vector.

Next, the activity of the resulting NKAR(28.z)-NK-92 cells was investigated in the absence or presence of NKAB-ErbB2 against MDA-MB453 breast cancer cells which express elevated levels of ErbB2 and various NKG2D ligands (see FIG. 2). As observed before for NKAR-NK-92, NKAR(28.z)-NK-92 cells already displayed increased lysis of MDA-MB453 cells in the absence of NKAB-ErbB2 when compared to parental NK-92 cells (36% versus 0% of specific killing at an E/T ratio of 10:1). This cytotoxicity was markedly enhanced to more than 60% specific lysis in the presence of NKAB-ErbB2 (FIG. 16B), demonstrating that a bispecific NKAB antibody can readily cooperate with different NKAR chimeric activating receptor formats.

Combination of bispecific NKAB antibodies with lymphocytes co-expressing an NKG2D-based chimeric activating receptor together with IL-15: Cytotoxic lymphocytes such as NK, NKT and T cells are dependent on cytokines such as IL-2 or IL-15 for growth and activity (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012)). These may be provided in the context of bispecific NKAB antibodies by introducing IL-15 or an IL-15 superagonist into the antibody molecule as described above, or by co-expressing such cytokines together with an NKG2D-based chimeric activating receptor in gene-modified immune effector cells. To test this, the initial NKAR vector pS-NKAR-IEW was modified to include in addition sequences encoding wildtype IL-15, or the IL-15 superagonist RD-IL15, linked to the NKAR sequence via a Porcine Teschovirus self-cleaving peptide (P2A) sequence (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012) and Zhu, X. et al. Novel human interleukin-15 agonists. J Immunol 183, 3598-3607 (2009)) (FIG. 16A, middle and bottom panels). VSV-G pseudotyped lentiviral pS-NKAR/IL15-IEW and pS-NKAR/RD-IL15-IEW vector particles were used for transduction of continuously expanding human NK-92 cells as a clinically relevant model for NK cells (Zhang, C. et al. Chimeric antigen receptor-engineered NK-92 cells: An off-the-shelf cellular therapeutic for targeted elimination of cancer cells and induction of protective antitumor immunity. Front Immunol 8, 533 (2017)), and resulting NKAR/IL15-NK-92 and NKAR/RD-IL15-NK-92 cells were enriched by flow cytometric cell sorting as described above for the initial NKAR vector.

Using NKAR/RD-IL15-NK-92 cells as an example for lymphocytes co-expressing an NKG2D-based CAR and IL-15, the activity of the cells was investigated in the absence or presence of NKAB-ErbB2 against MDA-MB453 breast cancer cells which express elevated levels of ErbB2 and various NKG2D ligands (see FIG. 2). As observed before for NKAR-NK-92 and NKAR(28.z)-NK-92, NKAR/RD-IL15-NK-92 cells already displayed increased lysis of MDA-MB453 cells in the absence of NKAB-ErbB2 when compared to parental NK-92 cells (31% versus 8% of specific killing at an E/T ratio of 10:1). This cytotoxicity was markedly enhanced to 60% specific lysis in the presence of NKAB-ErbB2 (FIG. 16C), demonstrating that a bispecific NKAB antibody can readily cooperate with effector lymphocytes co-expressing an NKG2D-based activating receptor and an IL-15 molecule. Furthermore, the IL-15 molecule co-expressed together with an NKG2D-based activating receptor directly supports the survival and growth of the respective immune effector cells (shown for NKAR/RD-IL15-NK-92 cells in FIG. 16D).

### Material and Methods:

Cells and culture conditions: Human MDA-MB453, MDA-MB468, and JIMT-1 breast carcinoma cells, LNT-229 glioblastoma cells, HEK 293T embryonic kidney cells (all ATCC, Manassas, VA), and murine B16/ErbB2 melanoma (Xu, Y., Darcy, P. K. & Kershaw, M. H. Tumor-specific dendritic cells generated by genetic redirection of Toll-like receptor signaling against the tumor-associated antigen, erbB2. Cancer Gene Ther 14, 773-780 (2007)) and GL261/ErbB2 glioblastoma cells (Zhang, C. et al. ErbB2/HER2-specific NK cells for targeted therapy of glioblastoma. J Natl Cancer Inst 108 (2016)) were cultured in DMEM medium (Lonza, Cologne, Germany). Human K562 erythroleukemia cells (ATCC) were grown in RPMI 1640 medium (Lonza). All media were supplemented with 10% heat-inactivated FBS, 2 mmol/L L-glutamine, 100 U/mL penicillin, 100 μg/mL streptomycin (Life Technologies, Darmstadt, Germany). Medium for GL261/ErbB2 cells in addition contained 0.4 mg/mL G418. Human NK-92 cells (ATCC) were propagated in X-VIVO 10 medium (Lonza) supplemented with 5% heat-inactivated human plasma (German Red Cross Blood Donation Service Baden-Württemberg—Hessen, Frankfurt, Germany) and 100 IU/mL IL-2 (Proleukin; Novartis Pharma, Nürnberg, Germany). For viability assays, NK-92 cells were washed, resuspended at a density of 2.5×10⁵ cells/mL in X-VIVO 10 medium with or without 100 IU/mL IL-2, and cultured for up to 7 days. Viability was analyzed by counting viable cells at different time points using trypan blue exclusion.

Peripheral blood NK cells of healthy donors were isolated from buffy coats by Ficoll-Hypaque density gradient centrifugation using the RosetteSep human NK cell enrichment cocktail (STEMCELL Technologies, Cologne, Germany) according to the manufacturer's instructions. Purity of the enriched NK cells was confirmed by flow cytometric analysis using BV421-conjugated anti-CD56 and PE-conjugated anti-CD3 antibodies (BD Biosciences, Heidelberg, Germany), and ranged between 83-96%. For ex vivo expansion, typically 1×10⁶ purified NK cells were cultured for up to 3 weeks in X-VIVO 10 growth medium (Lonza) supplemented with 5% heat-inactivated human plasma, 500 IU/mL IL-2 and 50 ng/ml IL-15 (PeproTech, Hamburg, Germany). Cells were maintained at a density of 1-2×10⁶ cells/mL throughout the culture period with half medium change every 2-3 days.

Human Raji Burkitt lymphoma cells were maintained in RPMI 1640 medium (Lonza). Human Mz-Mel-2 melanoma cells were cultured in DMEM medium (Lonza). All media were supplemented with 10% heat-inactivated FBS, 2 mmol/L L-glutamine, 100 U/mL penicillin, 100 μg/mL streptomycin (Life Technologies).

Expression and purification of bispecific NKAB-ErbB2 antibody: The IgG₄-based NKAB-ErbB2 sequence was designed by in silico assembly of an immunoglobulin heavy chain signal peptide, a single chain fragment variable (scFv) of NKG2D-specific antibody KYK-2.0 (Kwong, K. Y., Baskar, S., Zhang, H., Mackall, C. L. & Rader, C. Generation, affinity maturation, and characterization of a human anti-human NKG2D monoclonal antibody with dual antagonistic and agonistic activity. J Mol Biol 384, 1143-1156 (2008)), hinge, CH₂ and CH₃ domains of human IgG₄ (UniProtKB—P01861; amino acid residues 104-327), a (G₄S)₂ linker, and the ErbB2- specific SCFv(FRP5) antibody fragment (Wels, W. et al. Construction, bacterial expression and characterization of a bifunctional single-chain antibody-phosphatase fusion protein targeted to the human erbB-2 receptor. Biotechnology (N Y) 10, 1128-1132 (1992) and Schönfeld, K. et al. Selective inhibition of tumor growth by clonal NK cells expressing an ErbB2/HER2-specific chimeric antigen receptor. Mol Ther 23, 330-338 (2015)). For the similar NKAB-ErbB2 (IgG₁) molecule the hinge, CH₂ and CH₃ domains of human IgG₁ were used instead of IgG₄ (UniProtKB—P01857; amino acid residues 99-330). To test whether also other alternative designs of the bispecific molecule are functional, the scFv antibody fragments within the IgG4-based NKAB-ErbB2 sequence were exchanged resulting in NKAB-ErbB2 (rev) with reverse orientation of the binding domains. For comparison, the modified NKAB-ErbB2 (C106S, C109S) sequence was designed wherein the cysteine residues 106 and 109 within the IgG₄ hinge region (numbering according to UniProtKB—P01861) are replaced by serine residues to prevent formation of intermolecular disulfide bridges and homodimerization of the resulting protein. Codon-optimized fusion genes were de novo synthesized (GeneArt, Thermo Fisher Scientific, Darmstadt, Germany) and inserted into mammalian expression vector pcDNA3, resulting in plasmids pcDNA3-NKAB-ErbB2, pcDNA3-NKAB-ErbB2 (IgG₁), pcDNA3-NKAB-ErbB2 (rev) and pcDNA3-NKAB-ErbB2 (C106S, C109S). Following a similar strategy, as a control monospecific mini-antibody FRP5-Fc was generated, which encompasses an immunoglobulin heavy chain signal peptide, the ErbB2-specific scFv(FRP5) antibody fragment, and hinge, CH₂ and CH₃ domains of human IgG₁. Recombinant antibodies were expressed in transiently transfected HEK 293T cells and purified from culture supernatant by affinity chromatography using a HiTrap Protein-G column on an ÄKTA FPLC system (GE Healthcare Europe, Freiburg, Germany). Purity and integrity of NKAB antibodies was determined by SDS-PAGE and Coomassie staining, or immunoblotting with HRP-conjugated anti-human IgG antibody (Sigma-Aldrich, Munich, Germany). Protein concentrations were determined using a Nanodrop 1000 spectrophotometer (Thermo Fisher Scientific).

Expression and purification of additional bispecific NKAB antibodies: Sequences encoding bispecific NKAB antibodies targeting epidermal growth factor receptor (EGFR), the EGFR mutant form EGFRvIII, the disialoganglioside GD2, programmed death-ligand 1 (PD-L1), or the differentiation antigens CD19 and CD20 were generated by replacing the ErbB2-specific scFv(FRP5) antibody fragment in the IgG₄-based NKAB-ErbB2 sequence with codon-optimized EGFR-specific scFv(R1) or scFv(225), EGFRvIII-specific scFv(MR1-1) (Genßler, S. et al. Dual targeting of glioblastoma with chimeric antigen receptor-engineered natural killer cells overcomes heterogeneity of target antigen expression and enhances antitumor activity and survival. Oncoimmunology 5, e1119354 (2016)), GD2-specific scFv(14.18) (Esser, R. et al. NK cells engineered to express a GD2 -specific antigen receptor display built-in ADCC-like activity against tumour cells of neuroectodermal origin. J Cell Mol Med 16, 569-581 (2012)), atezolizumab-derived PD-L1-specific scFv (Chatterjee, S. et al. A humanized antibody for imaging immune checkpoint ligand PD-L1 expression in tumors. Oncotarget 7, 10215-10227 (2016)), CD19-specific scFv(63) (Oelsner, S. et al. Chimeric antigen receptor-engineered cytokine-induced killer cells overcome treatment resistance of pre-B-cell acute lymphoblastic leukemia and enhance survival. Int J Cancer 139, 1799-1809 (2016).), or CD20-specific scFv(Leu-16) antibody sequences (Müller, T. et al. Expression of a CD20-specific chimeric antigen receptor enhances cytotoxic activity of NK cells and overcomes NK-resistance of lymphoma and leukemia cells. Cancer Immunol Immunother 57, 411-423 (2008)). NKAB antibody sequences encompassing in addition interleukin-15 or the IL-15 superagonist RD-IL15 were generated by inserting codon-optimized IL-15 or RD-IL15 sequences between the immunoglobulin heavy chain signal peptide and the target-cell specific N-terminal scFv antibody fragment of NKAB-ErbB2 and the IgG₄-based NKAB antibody sequences described above (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012) and Zhu, X. et al. Novel human interleukin-15 agonists. J Immunol 183, 3598-3607 (2009)). Respective recombinant NKAB, NKAB/IL15 and NKAB/RD-IL15 antibodies were expressed and purified as described for NKAB-ErbB2.

Expression of lineage markers, NKG2D and natural cytotoxicity receptors: Expression of NKG2D and lineage markers by peripheral blood mononuclear cells (PBMCs) from healthy donors was assessed by staining with PE-conjugated anti-NKG2D (Miltenyi Biotec, Bergisch Gladbach, Germany), BV421-conjugated anti-CD56, and APC-conjugated anti-CD3 (BD Biosciences) antibodies. For phenotypic characterization of ex vivo expanded primary NK cells, cells were stained with BV421-conjugated anti-CD56, PE-conjugated anti-CD3, Alexa Fluor 647-conjugated anti-CD16, PE-conjugated anti-NKp30, Alexa Fluor 647-conjugated anti-NKp44 (all BD Biosciences), PE-conjugated anti-NKG2D, and APC-conjugated anti-NKp46 (both Miltenyi Biotec) antibodies. All staining procedures were performed in the presence of a human Fc receptor blocking agent (BD Biosciences). Expression of NK-cell activating receptors by NK-92 and NKAR-NK-92 cells was determined using PE-conjugated anti-NKG2D, PE-conjugated anti-NKp30, APC-conjugated anti-NKp46 (all Miltenyi Biotec), and APC-conjugated anti-NKp44 (R&D Systems, Wiesbaden-Nordenstadt, Germany) antibodies. Flow cytometric analysis was performed with FACSCanto II or BD LSRFortessa flow cytometers (BD Biosciences), and data were analyzed using FACSDiva or FlowJo software (Version 10.0.7; FlowJo, Ashland, OR).

Generation of NKAR-expressing effector cells: The NKG2D-based chimeric activating receptor NKAR consists of an immunoglobulin heavy chain signal peptide, the NKG2D extracellular domain (UniProtKB—P26718; amino acid residues 82-216), a (G₄S)₂ linker, a Myc-tag and a modified CD8α hinge region (Schönfeld, K. et al. Selective inhibition of tumor growth by clonal NK cells expressing an ErbB2/HER2-specific chimeric antigen receptor. Mol Ther 23, 330-338 (2015)), followed by CD3ζ transmembrane and intracellular domains. The codon-optimized NKAR sequence was de novo synthesized (GeneArt, Thermo Fisher Scientific) and inserted into lentiviral transfer plasmid pHR'SIN-cPPT-SIEW (pSIEW) upstream of IRES and EGFP sequences (Demaison, C. et al. High-level transduction and gene expression in hematopoietic repopulating cells using a human immunodeficiency [correction of imunodeficiency] virus type 1-based lentiviral vector containing an internal spleen focus forming virus promoter. Hum Gene Ther 13, 803-813 (2002)), yielding vector pS-NKAR-IEW. VSV-G pseudotyped vector particles were produced using HEK 293T cells, and NK-92 cells were transduced as described (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012)). NKAR-positive cells were enriched by flow cytometric cell sorting with a FACSAria fluorescence-activated cell sorter (BD Biosciences), with selection based on EGFP expression and enhanced NKG2D signals detected with anti-NKG2D antibody (Clone 149810, R&D Systems) followed by APC-coupled secondary antibody (Dianova, Hamburg, Germany). NKAR expression by sorted cells was confirmed by SDS-PAGE of cell lysates and immunoblotting with anti-CD3ζ(6B10.2) or anti-CD8α antibodies (H-160; both Santa Cruz Biotechnology, Heidelberg, Germany), followed by HRP-conjugated secondary antibody and chemiluminescent detection. Similarly, CAR-engineered primary T cells were derived by lentiviral transduction with the NKAR construct. Interaction of NKAR with soluble MICA was investigated by flow cytometry with recombinant His-tagged human MICA (Biozol, Eching, Germany) followed by APC-conjugated anti-His-tag antibody (BioLegend, Koblenz, Germany).

Generation of effector cells expressing alternative NKAR formats: The NKG2D-based second-generation chimeric activating receptor NKAR(28.z) consists of an immunoglobulin heavy chain signal peptide, the NKG2D extracellular domain (UniProtKB—P26718; amino acid residues 82-216), a (G₄S)₂ linker, a Myc-tag and a modified CD8α hinge region (Schönfeld, K. et al. Selective inhibition of tumor growth by clonal NK cells expressing an ErbB2/HER2-specific chimeric antigen receptor. Mol Ther 23, 330-338 (2015)), followed by CD28 transmembrane and intracellular domains and the intracellular domain of CD3ζ. The codon-optimized NKAR(28.z) sequence was de novo synthesized (GeneArt, Thermo Fisher Scientific) and inserted into lentiviral transfer plasmid pHR'SIN-cPPT-SIEW (pSIEW) upstream of IRES and EGFP sequences (Demaison, C. et al. High-level transduction and gene expression in hematopoietic repopulating cells using a human immunodeficiency [correction of imunodeficiency] virus type 1-based lentiviral vector containing an internal spleen focus forming virus promoter. Hum Gene Ther 13, 803-813 (2002)), yielding vector pS-NKAR(28.z)-IEW. Lentiviral transfer plasmids pS-NKAR_IL15-IEW and pS-NKAR_RD-IL15-IEW were generated by fusing sequences encoding IL-15 or the IL-15 superagonist RD-IL15 to the 3′-end of the NKAR sequence via a Porcine Teschovirus self-cleaving peptide (P2A) sequence (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012) and Zhu, X. et al. Novel human interleukin-15 agonists. J Immunol 183, 3598-3607 (2009)). VSV-G pseudotyped vector particles were produced, NK-92 cells were transduced, and chimeric activating receptor-expressing cells were enriched as described above for the initial NKAR vector.

Cytotoxicity assays: Cytotoxicity of NK-92 cells and primary lymphocytes towards tumor cells was analyzed in FACS-based assays as described (Sahm, C., Schonfeld, K. & Wels, W. S. Expression of IL-15 in NK cells results in rapid enrichment and selective cytotoxicity of gene-modified effectors that carry a tumor-specific antigen receptor. Cancer Immunol Immunother 61, 1451-1461 (2012)). Briefly, target cells were labeled with calcein violet AM (CV) (Molecular Probes, Invitrogen, Karlsruhe, Germany) and incubated with effector cells at various effector to target (E/T) ratios for 3 hours at 37° C. in the presence or absence of bispecific antibodies. Then 150 μL of a 1 μg/mL propidium iodide (PI) solution were added to each sample before flow cytometric analysis in a FACSCanto II flow cytometer (BD Biosciences). Dead target cells were identified as CV and PI double positive. Spontaneous target cell lysis in the absence of effector cells was subtracted to calculate specific cytotoxicity. Data were analyzed using FACSDiva software (BD Biosciences). For competition experiments, soluble MICA-Fc fusion protein (R&D Systems) or recombinant human IgG₄ protein (Biozol) were added to the cultures. Cytokine release by NK-92 and NKAR-NK-92 cells was measured using a BD Cytometric Bead Array (BD Biosciences) as described (Oelsner, S. et al. Chimeric antigen receptor-engineered cytokine-induced killer cells overcome treatment resistance of pre-B-cell acute lymphoblastic leukemia and enhance survival. Int J Cancer 139, 1799-1809 (2016)).

In vivo tumor model: Six to 8 week old female C57BL/6 mice were used for a syngeneic GL261/ErbB2 murine glioblastoma model. Mice were inoculated with 1×10⁶ tumor cells at the right flank. Seven days later, the animals were treated by peritumoral injection of 1×10⁷ NKAR-NK-92 or parental NK-92 cells in 200 μL of injection medium, with or without addition of 5 μg of NKAB-ErbB2 antibody. Treatment was repeated twice per week for 3 weeks. Tumor growth was followed by caliper measurements and tumor volumes were calculated using the formula: length×(width)²×0.5. The experiments were terminated when the defined study endpoints were reached. All animal experiments were approved by the responsible government committee (Regierungspräsidium Darmstadt, Darmstadt, Germany), and were conducted according to the applicable guidelines and regulations.

1. A method of treating a disease in a subject in need thereof, said method comprising administering to the subject a binding molecule and an immune cell, wherein the binding molecule is at least bispecific and comprises at least a first binding domain and a second binding domain, wherein:
(a) the first binding domain is capable of binding to NKG2-D type II integral membrane protein (NKG2D, SEQ ID NO: 1); and
(b) the second binding domain is capable of binding to an antigenic target protein expressed on or in a cell associated with the disease in the subject;
and wherein the immune cell comprises a chimeric antigen receptor (CAR) comprising an extracellular NKG2D sequence (NKAR).

2. The method of claim 1, wherein the first binding domain competitively binds to NKG2D compared with an NKG2D-ligand, and wherein the NKG2D-ligand is for example a MHC class I polypeptide-related sequence A (MICA) or soluble MICA (sMICA).

3. The method of claim 1, wherein the first binding domain and/or the second binding domain comprises one or more binding domains of an antibody, such as an scFv construct.

4. The method of claim 1, wherein the first binding domain and the second binding domain are linked to each other by a protein linker comprising one or more human antibody constant domains, such as preferably of an IgG (such as IgG₁ or IgG₄), for example they are linked via human IgG₁ or IgG₄ hinge, CH₂ and CH₃.

5. The method of claim 1, further comprising an interleukin-15 domain fused to either the first binding domain and/or the second binding domain, wherein the interleukin-15 domain optionally is an interleukin-15 agonist.

6. The method of claim 1, wherein the immune cell is a cytotoxic cell, such as a cell expressing NKG2D and preferably is a T cell, natural killer (NK) cell, or NKT cell.

7. The method of claim 1, wherein the immune cell further comprises an interleukin-15, or an interleukin-15 agonist.

8. The method of claim 1, wherein the NKAR further comprises:
(a) a hinge region such as a CD8α hinge region; and/or
(b) a transmembrane domain such as a transmembrane domain from CD3ζ or CD28; and
(c) an intracellular signaling domain such as an intracellular domain from CD3ζ; and optionally
(d) one or more intracellular costimulatory domains such as a CD28 intracellular domain.

9. The method of claim 1, wherein the disease is a proliferative disease, preferably selected from a cancer disease, such as a cancer, for example lung cancer, breast cancer, colorectal cancer, gastric cancer, liver cancer, pancreatic cancer, ovarian cancer, melanoma, kidney cancer, head and neck cancer, brain cancer, skin cancer, myeloma, lymphoma, or leukemia, or wherein the disease is an infectious disease, such as a viral infection, for example an infection with a virus selected from HIV, HPV, HBV, HCV, EBV, CMV, Influenza Virus, SARS-COV-1, or SARS-COV-2, preferably, wherein the proliferative disease is a cancer positive for an expression of the antigenic target protein, or wherein the infectious disease is a viral infection positive for an expression of the antigenic target protein.

10. A binding molecule which is at least bispecific comprising at least a first and a second binding domain, wherein
(a) the first binding domain is capable of binding to NKG2-D type II integral membrane protein (NKG2D, SEQ ID NO: 1); and
(b) the second binding domain is capable of binding to an antigenic target protein expressed on or in a cell associated with a disease in the subject;
wherein the first and the second binding domain are antibody scFv constructs and are linked to each other via human IgG₁ or IgG₄ hinge, CH₂ and CH₃ domains.

11. The binding molecule of claim 10, further comprising an interleukin-15 domain fused to either the first and/or the second binding domain, wherein the interleukin-15 domain optionally is an interleukin-15 agonist.

12. The binding molecule of claim 10, wherein the binding molecule comprises two first binding domains and two second binding domains.

13. The binding molecule of claim 12, wherein the binding molecule comprises two antibody scFv constructs as first binding domains and two antibody scFv constructs as second binding domains, connected by an antibody Fc region (dimeric scFv2-Fc format).

14. A method of treating a disease in a subject in need thereof, said method comprising administering to the subject an immune cell and a binding molecule, wherein the immune cell expresses an immune cell receptor, wherein the immune cell receptor is NKG2D, or a derivative thereof such as a CAR (NKAR), and wherein the immune cell optionally further expresses an interleukin-15, or an interleukin-15 agonist, and wherein the binding molecule comprises is at least bispecific and comprises at least a first binding domain and a second binding domain, wherein:
(a) the first binding domain is capable of binding to NKG2-D type II integral membrane protein (NKG2D, SEQ ID NO: 1); and
(b) the second binding domain is capable of binding to an antigenic target protein expressed on or in a cell associated with the disease in the subject.

15. The method of claim 14, wherein the immune cell is a cytotoxic cell, such as a cell expressing NKG2D and preferably is a T cell, natural killer (NK) cell, or NKT cell.

16. The method of claim 14, wherein the immune cell is an autologous or allogeneic immune cell, and preferably is genetically engineered to have an increased expression of NKG2D.

17. An isolated nucleic acid, comprising one or more sequences encoding the binding molecule as defined in claim 1.

18. A recombinant host cell, comprising the nucleic acid of claim 17.

19. A pharmaceutical composition or package comprising:
(a) (i) the binding molecule as defined in claim 1; or (ii) an isolated nucleic acid encoding said binding molecule, or (iii) a recombinant host cell comprising a nucleic acid encoding said binding molecule; and/or
(b) an immune cell expressing an immune cell receptor, or an immune cell receptor, wherein the immune cell receptor is NKG2D, or a derivative thereof such as a CAR (NKAR), and wherein the immune cell optionally further expresses an interleukin-15, or an interleukin-15 agonist;
together with a pharmaceutically acceptable carrier, stabiliser and/or excipient.
